(19) United States
(12) Patent Application Publication
(10) Pub. No.: US 2024/0109836 A1
(43) Pub. Date: Apr. 4, 2024

(54) NON-HALLUCINOGENIC ARIADNE ANALOGS FOR TREATMENT OF NEUROLOGICAL AND PSYCHIATRIC DISORDERS

(71) Applicant: The Trustees of Columbia University in the City of New York, New York, NY (US)

(72) Inventors: Michael J. Cunningham, Asheville, NC (US); Dalibor Sames, New York, NY (US); Benjamin Bechand, New York, NY (US)

(73) Assignee: The Trustees of Columbia University in the City of New York, New York, NY (US)

(21) Appl. No.: 18/468,258

(22) Filed: Sep. 15, 2023

Related U.S. Application Data
(63) Continuation of application No. PCT/US2022/021604, filed on Mar. 23, 2022.
(60) Provisional application No. 63/215,248, filed on Jun. 25, 2021.
(60) Provisional application No. 63/165,605, filed on Mar. 24, 2021.

(51) Int. Cl. C07C 217/60; C07C 255/59; C07C 317/32; C07C 323/32; C07D 305/06; C07F 7/08

(52) U.S. Cl. CPC C07C 217/60; C07C 255/59; C07C 317/32; C07C 323/32; C07D 305/06; C07F 7/081

(57) ABSTRACT

The present invention provides a compound having the structure:

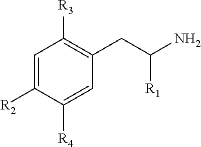

wherein
R₁ is —(C₂-C₁₂ alkyl), -(alkenyl), -(alkynyl), -(cycloalkyl);
R₂ is H, halogen, —NO₂, —CN, —CF₃, —CF₂H, —CH₂OCH₃, —CF₂CH₃, —CF₂OCH₃, -(alkyl), -(alkenyl), -(alkynyl), -(cycloalkyl), -(cycloalkylalkyl), -(heteroalkyl), -(heterocycle), -(heterocycloalkyl), -(aryl), -(heteroaryl), -(hydroxyalkyl), -(haloalkyl), -(alkylaryl), —OH, —O-(alkyl), —O-(alkenyl), —O-(alkynyl), —O-(haloalkyl), —O-(aryl), —O-(heteroaryl), —OCF₃, SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —N-(alkyl)₂, NH-(aryl), —NH-(heteroaryl), —CO₂H, —CO₂-(alkyl), —C(O)—NH₂, —C(O)—NH-(alkyl), —C(O)—NH-(aryl), —SO₂CH₃ or —Si(CH₃) ₃;
R₃ is —OCH₃, —OCH₂CH₃, —F or —Cl; and
R₄ is —OCH₃, —OCH₂CH₃ or —SCH₃;
wherein when R₁ is —CH₂CH₃, R₃ is —OCH₃, and R₄ is —OCH₃, then R₂ is other than H, —CH₃, —CH₂CH₃, —CH₂CH₂CH₃, —CH(CH₃)₂, —CH₂CH₂CH₂CH₃, —CH₂OH, —CH(OH) CH₃, —OH, —OCH₂CH₃, —OCH₂CH₂CH₃, —OCH(CH₃)₂, —SCH₃, —SCH₂CH₃, —SCH₂CH₂CH₃, —NO₂, —NH₂, —F, —Cl, —Br or —I,
   or a pharmaceutically acceptable salt thereof.

This application claims priority of U.S. Provisional Application No. 63/215,248, filed Jun. 25, 2021, and U.S. Provisional Application No. 63/165,605, filed Mar. 24, 2021, the contents of which are hereby incorporated by reference.

Throughout this application, certain publications are referenced in parentheses. Full citations for these publications may be found immediately preceding the claims. The disclosures of these publications in their entireties are hereby incorporated by reference into this application in order to describe more fully the state of the art to which this invention relates.

### BACKGROUND OF THE INVENTION

The effects of the classic psychedelics currently studied for use in a variety of indications are mediated by engagement of serotonin receptors. Specifically, activation of the serotonin 2A (5HT2A) receptor is responsible for not only therapeutic effects but also the associated problematic hallucinogenesis (Nichols, D. E. 2016). Although there is a renewed interest in harnessing the transient altered states induced by psychedelics for therapeutic purposes, there is a need for agents with no or limited hallucinogenic effects. The practice of taking very small amounts of psychedelic 5HT2A receptor agonists (“microdosing”) has gained popularity as a means of improving mood, cognition or perceived well-being (Kuypers, K. P. et al. 2019). However, clinical efficacy of this approach remains to be demonstrated in rigorous controlled clinical studies, and is likely to be marginal (compared to placebo) owing to the low engagement of the 5HT2A receptor. Further, despite the goal of dosing below the perceptual threshold to avoid impairment of normal behavior and functioning, there remains the real risk of dosing outside of this safe range into the realm of fully psychedelic activity. It is imperative that compounds are made available that deliver the therapeutic benefits of 5HT2A receptor agonists while limiting the potential for adverse events. Development of such compounds would present new options for effective maintenance therapies for various indications outside of clinical settings (i.e. as take-home medicines).

Depression affects more than 300 million people worldwide and is associated with an economic burden of over $200 billion dollars. Psilocybin, the leading 5HT2A agonist clinical candidate has demonstrated marked efficacy in proof-of-concept depression and anxiety clinical studies (Davis, A. K. et al. 2020). While psilocybin's effect is rapid, large and long-lasting, it also has drawbacks. Due to its powerful psychedelic properties, it must be taken in a clinical setting under the supervision of therapists, limiting its wide use. The non-psychedelic analogs may be used for treatment of psychiatric disorders such as depression (Griffiths, R. R. et al. 2016; Davis, A. K. et al. 2020), PTSD, OCD, bipolar disorder, ADHD (Partyka, R. A. et al. 1978), schizophrenia (Partyka, R. A. et al. 1978), and substance use disorders (Bogenschutz, M. P. et al. 2015; Garcia-Romeu, A. et al. 2014; Cameron, L. P. et al. 2021), as well as neurological disorders such as Parkinson's disease (Partyka, R. A. et al. 1978), dementias including Alzheimer's disease (Partyka, R. A. et al. 1978), cluster headaches (Sewell, R. A. et al. 2006) and various ocular conditions (Feng, Z. et al. 2007; Foster, T. P. & Nichols, C. D. 2018; May, J. et al. 2000).

The search for non-hallucinogenic psychedelic analogs have begun shortly after the discovery of LSD at Sandoz. This effort was guided by the concept of identifying LSD derivatives with serotonin blocking effects or serotonin antagonists (Hofmann, A. 2009). Another example is the identification of Ariadne [2-amino-1-(2,5-dimethoxy-4-methylphenyl)butane), an analog of psychedelic phenylalkylamine, that showed no hallucinogenic effect at therapeutically effective doses in animals and humans. However, the mechanism of action of Ariadne has not been previously elucidated (Standridge, R. T. et al. 1976). In this invention we define “non-hallucinogenic psychedelics” as 5HT2A receptor agonists that induce no or limited hallucinogenic effects at pharmacologically and physiologically meaningful engagement of the target receptor.

Psychedelic phenylalkylamines are a class of compounds that have been used by humans for thousands of years in the form of cacti that contain mescaline, hordenine and lophophine and other alkaloids (Terry, M. et al. 2006). Medicinal chemistry efforts around amphetamines converged with human interest in these compounds in the twentieth century, producing numerous interesting novel psychoactive compounds (Alles, G. A. 1959). Of considerable interest has been the 2,5-dimethoxy amphetamines and phenethylamines which maintain varying psychoactive effects across a range of substituents at the 4-position of the aromatic ring (Shulgin, A. T. 1968; Barfknecht, C. F. & Nichols, D. E. 1971).

We here show that Ariadne acts as a selective 5HT2A receptor agonist and thus provide a mechanism of action of this compound. We also introduce novel analogs that demonstrate increased selectivity for 5HT2A receptor over 5HT2B and other receptors (and neurotransmitter transporters). Despite the agonism at 5HT2A receptor these compounds exert no or limited hallucinogenic effects at therapeutically effective doses.

### SUMMARY OF THE INVENTION

The present invention provides a compound having the structure:

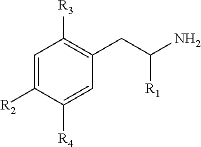

wherein
R₁ is —(C₂-C₁₂ alkyl), -(alkenyl), -(alkynyl), -(cycloalkyl);
R₂ is H, halogen, —NO₂, —CN, —CF₃, —CF₂H, —CH₂OCH₃, —CF₂CH₃, —CF₂OCH₃, -(alkyl), -(alkenyl), -(alkynyl), -(cycloalkyl), -(cycloalkylalkyl), -(heteroalkyl), -(heterocycle), -(heterocycloalkyl), -(aryl), -(heteroaryl), -(hydroxyalkyl), -(haloalkyl), -(alkylaryl), —OH, —O-(alkyl), —O-(alkenyl), —O-(alkynyl), —O-(haloalkyl), —O-(aryl), —O-(heteroaryl), —OCF₃, SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —N-(alkyl)₂, NH-(aryl), —NH-(heteroaryl), —CO₂H, —CO₂-(alkyl), —C(O)—NH₂, —C(O)—NH-(alkyl), —C(O)—NH-(aryl), —SO₂CH₃ or —Si(CH₃) ₃;
R₃ is —OCH₃, —OCH₂CH₃, —F or —Cl; and
R₄ is —OCH₃, —OCH₂CH₃ or —SCH₃;
wherein when R₁ is —CH₂CH₃, R₃ is —OCH₃, and R₄ is —OCH₃, then R₂ is other than H, —CH₃, —CH₂CH₃, —CH₂CH₂CH₃, —CH(CH₃)₂, —CH₂CH₂CH₂CH₃, —CH₂OH, —CH(OH)CH₃, —OH, —OCH₂CH₃, —OCH₂CH₂CH₃, —OCH(CH₃)₂, —SCH₃, —SCH₂CH₃, —SCH₂CH₂CH₃, —NO₂, —NH₂, —F, —Cl, —Br or —I,
   or a pharmaceutically acceptable salt thereof.

The present invention also provides a compound having the structure:

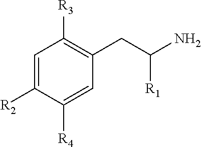

wherein
R₁ is —(C₃-C₁₂ alkyl), -(alkenyl), -(alkynyl), -(cycloalkyl);
R₂ is H, halogen, —NO₂, —CN, —CF₃, —CF₂H, —CH₂OCH₃, —CF₂CH₃, —CF₂OCH₃, -(alkyl), -(alkenyl), -(alkynyl), -(cycloalkyl), -(cycloalkylalkyl), -(heteroalkyl), -(heterocycle), -(heterocycloalkyl), -(aryl), -(heteroaryl), -(hydroxyalkyl), -(haloalkyl), -(alkylaryl), —OH, —O-(alkyl), —O-(alkenyl), —O-(alkynyl), —O-(haloalkyl), —O-(aryl), —O-(heteroaryl), —OCF₃, SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —N-(alkyl)₂, NH-(aryl), —NH-(heteroaryl), —CO₂H, —CO₂-(alkyl), —C(O)—NH₂, —C(O)—NH-(alkyl), —C(O)—NH-(aryl), —SO₂CH₃ or —Si(CH₃)₃;
R₃ is —OCH₃, —OCH₂CH₃, —F or —Cl; and
R₄ is —OCH₃, —OCH₂CH₃ or —SCH₃,
   or a pharmaceutically acceptable salt thereof.

The present invention also provides a method of activating 5HT2A receptor in a subject comprising administering to a subject an effective amount of a compound having the structure:

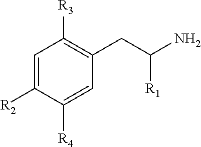

wherein
R₁ is —(C₂-C₁₂ alkyl), -(alkenyl), -(alkynyl), -(cycloalkyl)
R₂ is H, halogen, —NO₂, —CN, —CF₃, —CF₂H, —CH₂OCH₃, —CF₂CH₃, —CF₂OCH₃, -(alkyl), -(alkenyl), -(alkynyl), -(cycloalkyl), -(cycloalkylalkyl), -(heteroalkyl), -(heterocycle), -(heterocycloalkyl), -(aryl), -(heteroaryl), -(hydroxyalkyl), -(haloalkyl), -(alkylaryl), —OH, —O-(alkyl), —O-(alkenyl), —O-(alkynyl), —O-(haloalkyl), —O-(aryl), —O-(heteroaryl), —OCF₃, SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —N-(alkyl)₂, NH-(aryl), —NH-(heteroaryl), —CO₂H, —CO₂-(alkyl), —C(O)—NH₂, —C(O)—NH-(alkyl), —C(O)—NH-(aryl), —SO₂CH₃ or —Si(CH₃) ₃;
R₃ is —OCH₃, —OCH₂CH₃, —F or —Cl; and
R₄ is —OCH₃, —OCH₂CH₃, —SCH₃, —F or —Cl;
   or a pharmaceutically acceptable salt thereof, so as to thereby activate 5HT2A receptor in a subject.

### BRIEF DESCRIPTION OF THE FIGURES

FIG. 1. Forced swim test indication of antidepressant-like effects in mice. N=5 mice (saline), N=10 (imipramine as a positive control), N=9 mice (Ariadne cohort). Y-axis show the immobility time per each testing session. Drugs were administered subcutaneously (s.c.).

FIG. 2. Mouse head twitch response assay indicates lower psychedelic activity of Ariadne (32), 1-(2,5-dimethoxy-4-(trifluoromethyl)phenyl)butan-2-amine (29) and 1-(2,5-dimethoxy-4-(methoxymethyl)phenyl)butan-2-amine (38) vs positive control DOI (1-(4-iodo-2,5-dimethoxyphenyl)propan-2-amine). N=5 mice in each cohort. Y-axis shows number of head twitches in a 10-min period. Drugs were administered s.c.

### DETAILED DESCRIPTION OF THE INVENTION

The present invention provides a compound having the structure:

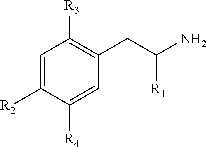

wherein
R₁ is —(C₂-C₁₂ alkyl), -(alkenyl), -(alkynyl), -(cycloalkyl);
R₂ is H, halogen, —NO₂, —CN, —CF₃, —CF₂H, —CH₂OCH₃, —CF₂CH₃, —CF₂OCH₃, -(alkyl), -(alkenyl), -(alkynyl), -(cycloalkyl), -(cycloalkylalkyl), -(heteroalkyl), -(heterocycle), -(heterocycloalkyl), -(aryl), -(heteroaryl), -(hydroxyalkyl), -(haloalkyl), -(alkylaryl), —OH, —O-(alkyl), —O-(alkenyl), —O-(alkynyl), —O-(haloalkyl), —O-(aryl), —O-(heteroaryl), —OCF₃, SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —N-(alkyl)₂, NH-(aryl), —NH-(heteroaryl), —CO₂H, —CO₂-(alkyl), —C(O)—NH₂, —C(O)—NH-(alkyl), —C(O)—NH-(aryl), —SO₂CH₃ or —Si(CH₃) ₃;
R₃ is —OCH₃, —OCH₂CH₃, —F or —Cl; and
R₄ is —OCH₃, —OCH₂CH₃ or —SCH₃;
wherein when R₁ is —CH₂CH₃, R₃ is —OCH₃, and R₄ is —OCH₃, then R₂ is other than H, —CH₃, —CH₂CH₃, —CH₂CH₂CH₃, —CH(CH₃)₂, —CH₂CH₂CH₂CH₃, —CH₂OH, —CH(OH)CH₃, —OH, —OCH₂CH₃, —OCH₂CH₂CH₃, —OCH(CH₃)₂, —SCH₃, —SCH₂CH₃, —SCH₂CH₂CH₃, —NO₂, —NH₂, —F, —Cl, —Br or —I,
   or a pharmaceutically acceptable salt thereof.

The present invention also provides a compound having the structure:

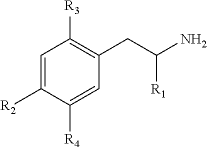

wherein
R₁ is —(C₃-C₁₂ alkyl), -(alkenyl), -(alkynyl), -(cycloalkyl);
R₂ is H, halogen, —NO₂, —CN, —CF₃, —CF₂H, —CH₂OCH₃, —CF₂CH₃, —CF₂OCH₃, -(alkyl), -(alkenyl), -(alkynyl), -(cycloalkyl), -(cycloalkylalkyl), -(heteroalkyl), -(heterocycle), -(heterocycloalkyl), -(aryl), -(heteroaryl), -(hydroxyalkyl), -(haloalkyl), -(alkylaryl), —OH, —O-(alkyl), —O-(alkenyl), —O-(alkynyl), —O-(haloalkyl), —O-(aryl), —O-(heteroaryl), —OCF₃, SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —N-(alkyl)₂, NH-(aryl), —NH-(heteroaryl), —CO₂H, —CO₂-(alkyl), —C(O)—NH₂, —C(O)—NH-(alkyl), —C(O)—NH-(aryl), —SO₂CH₃ or —Si(CH₃)₃;
R₃ is —OCH₃, —OCH₂CH₃, —F or —Cl; and
R₄ is —OCH₃, —OCH₂CH₃ or —SCH₃,
   or a pharmaceutically acceptable salt thereof.

The present invention also provides a compound having the structure:

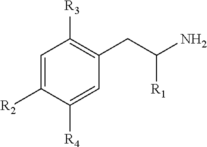

wherein
R₁ is —(C₂-C₁₂ alkyl), -(alkenyl), -(alkynyl), -(cycloalkyl)
R₂ is H, halogen, —NO₂, —CN, —CF₃, —CF₂H, -(alkyl), -(alkenyl), -(alkynyl), -(cycloalkyl), -(cycloalkylalkyl), -(heteroalkyl), -(heterocycle), -(heterocycloalkyl), -(aryl), -(heteroaryl), -(hydroxyalkyl), -(haloalkyl), -(alkylaryl), —OH, —O-(alkyl), —O-(alkenyl), —O-(alkynyl), —O-(haloalkyl), —O-(aryl), —O-(heteroaryl), —OCF₃, SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —N-(alkyl)₂, NH-(aryl), —NH-(heteroaryl), —CO₂H, —CO₂-(alkyl), —C(O)—NH₂, —C(O)—NH-(alkyl), —C(O)—NH-(aryl) or —Si(CH₃)₃;
R₃ is —OCH₃, —F or —Cl; and
R₄ is —OCH₃ or —SCH₃;
wherein when R₁ is —CH₂CH₃, R₃ is —OCH₃, and R₄ is —OCH₃, then R₂ is other than H, —CH₃, —CH₂CH₃, —CH₂CH₂CH₃, —CH(CH₃)₂, —CH₂CH₂CH₂CH₃, —CH₂OH, —CH(OH) CH₃, —OH, —OCH₂CH₃, —OCH₂CH₂CH₃, —OCH(CH₃)₂, —SCH₃, —SCH₂CH₃, —SCH₂CH₂CH₃, —NO₂, —NH₂, —F, —Cl, —Br or —I,
   or a pharmaceutically acceptable salt thereof.

The present invention also provides a compound having the structure:

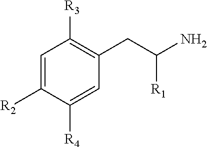

wherein
R₁ is —(C₃-C₁₂ alkyl), -(alkenyl), -(alkynyl), -(cycloalkyl);
R₂ is H, halogen, —NO₂, —CN, —CF₃, —CF₂H, -(alkyl), -(alkenyl), -(alkynyl), -(cycloalkyl), -(cycloalkylalkyl), -(heteroalkyl), -(heterocycle), -(heterocycloalkyl), -(aryl), -(heteroaryl), -(hydroxyalkyl), -(haloalkyl), -(alkylaryl), —OH, —O-(alkyl), —O-(alkenyl), —O-(alkynyl), —O-(haloalkyl), —O-(aryl), —O-(heteroaryl), —OCF₃, SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —N-(alkyl)₂, NH-(aryl), —NH-(heteroaryl), —CO₂H, —CO₂-(alkyl), —C(O)—NH₂, —C(O)—NH-(alkyl), —C(O)—NH-(aryl) or —Si(CH₃)₃;
R₃ is —OCH₃, —F or —Cl; and
R₄ is —OCH₃ or —SCH₃,
   or a pharmaceutically acceptable salt thereof.

In some embodiments, the compound wherein
R₁ is —(C₂-C₁₂ alkyl), -(alkenyl), -(alkynyl), -(cycloalkyl);
R₂ is H, —CN, —CF₃, —CH₂OCH₃, —CF₂CH₃, —CF₂OCH₃, -(alkyl), -(alkenyl), -(alkynyl), -(cycloalkyl), -(heterocycle), —S-(aryl), —SO₂CH₃ or —Si(CH₃)₃;
R₃ is —OCH₃, —OCH₂CH₃, —F or —Cl; and
R₄ is —OCH₃, —OCH₂CH₃ or —SCH₃.

In some embodiments, the compound wherein
R₁ is —(C₂-C₁₂ alkyl), -(alkenyl), -(alkynyl), -(cycloalkyl);
R₂ is H, —CN, —CF₃, -(alkyl), -(alkenyl), -(alkynyl), -(cycloalkyl), -(heterocycle), —S-(aryl) or —Si(CH₃)₃;
R₃ is —OCH₃, —F or —Cl; and
R₄ is —OCH₃ or —SCH₃.

In some embodiments, the compound wherein when R₁ is —CH₂CH₃, R₃ is —OCH₃, and R₄ is —OCH₃, then R₂ is other than H, —CH₃, —CH₂CH₃, —CH₂CH₂CH₃, —CH(CH₃)₂, —CH₂CH₂CH₂CH₃, —CH₂OH, —CH(OH) CH₃, —OH, —OCH₂CH₃, —OCH₂CH₂CH₃, —OCH(CH₃)₂, —SCH₃, —SCH₂CH₃, —SCH₂CH₂CH₃, —NO₂, —NH₂, —F, —Cl, —Br or —I.

In some embodiments, the compound wherein
R₁ is —(C₃-C₁₂ alkyl), -(alkenyl), -(alkynyl), -(cycloalkyl);
R₂ is H, —CN, —CF₃, —CH₂OCH₃, —CF₂CH₃, —CF₂OCH₃, -(alkyl), -(alkenyl), -(alkynyl), -(cycloalkyl), -(heterocycle), —S-(aryl), —SO₂CH₃ or —Si(CH₃)₃;
R₃ is —OCH₃, —OCH₂CH₃, —F or —Cl; and
R₄ is —OCH₃, —OCH₂CH₃ or —SCH₃.

In some embodiments, the compound wherein
R₁ is —(C₃-C₁₂ alkyl), -(alkenyl), -(alkynyl), -(cycloalkyl);
R₂ is H, —CN, —CF₃, -(alkyl), -(alkenyl), -(alkynyl), -(cycloalkyl), -(heterocycle), —S-(aryl) or —Si(CH₃)₃;
R₃ is —OCH₃, —F or —Cl; and
R₄ is —OCH₃ or —SCH₃.

In some embodiments, the compound wherein
R₁ is —CH₂CH₃, —CH₂CH₂CH₃, —CH₂CH═CH₂ or cyclopropyl.

In some embodiments, the compound wherein
R₁ is —CH₂CH₂CH₃, —CH₂CH═CH₂ or cyclopropyl.

In some embodiments, the compound wherein
R₂ is —CN, —CF₃, —CF₂H, —CH₂OCH₃, —CF₂CH₃, —CF₂OCH₃, —CH₃, —CCH, -cyclopropyl, -cyclobutyl, 2-oxetanyl, —S-(phenyl), —SO₂CH₃ or —Si(CH₃) ₃.

In some embodiments, the compound wherein
R₂ is —CN, —CF₃, —CH₃, —CCH, -cyclopropyl, -cyclobutyl, 2-oxetanyl, —S-(phenyl) or —Si(CH₃)₃.

In some embodiments, the compound wherein
R₃ is —OCH₃.

In some embodiments, the compound wherein
R₃ is —F.

In some embodiments, the compound wherein
R₃ is —Cl.

In some embodiments, the compound wherein
R₃ is —OCH₂CH₃.

In some embodiments, the compound wherein
R₄ is —OCH₃.

In some embodiments, the compound wherein
R₄ is —SCH₃.

In some embodiments, the compound wherein
R₄ is —OCH₂CH₃.

In some embodiments, the compound wherein
R₁ is —CH₂CH₃, —CH₂CH₂CH₃, —CH₂CH═CH₂ or cyclopropyl;
R₂ is —CN, —CF₃, —CH₂OCH₃, —CF₂CH₃, —CF₂OCH₃, —CH₃, —CCH, -cyclopropyl, -cyclobutyl, 2-oxetanyl, —S-(phenyl), —SO₂CH₃ or —Si(CH₃) ₃;
R₃ is —OCH₃, —OCH₂CH₃, F or Cl; and
R₄ is —OCH₃, —OCH₂CH₃ or —SCH₃.

In some embodiments, the compound wherein
R₁ is —CH₂CH₃, —CH₂CH₂CH₃, —CH₂CH═CH₂ or cyclopropyl;
R₂ is —CN, —CF₃, —CH₃, —CCH, -cyclopropyl, -cyclobutyl, 2-oxetanyl, —S-(phenyl) or —Si(CH₃)₃;
R₃ is —OCH₃, F or Cl; and
R₄ is —OCH₃ or —SCH₃.

In some embodiments, the compound wherein
R₁ is —CH₂CH₂CH₃, —CH₂CH═CH₂ or cyclopropyl;
R₂ is —CN, —CF₃, —CH₂OCH₃, —CF₂CH₃, —CF₂OCH₃, —CH₃, —CCH, -cyclopropyl, -cyclobutyl, 2-oxetanyl, —S-(phenyl), —SO₂CH₃ or —Si(CH₃) ₃;
R₃ is —OCH₃, —OCH₂CH₃, F or Cl; and
R₄ is —OCH₃, —OCH₂CH₃, or —SCH₃.

In some embodiments, the compound wherein
R₁ is —CH₂CH₂CH₃, —CH₂CH═CH₂ or cyclopropyl;
R₂ is —CN, —CF₃, —CH₃, —CCH, -cyclopropyl, -cyclobutyl, 2-oxetanyl, —S-(phenyl) or —Si(CH₃)₃;
R₃ is —OCH₃, F or Cl; and
R₄ is —OCH₃ or —SCH₃.

In some embodiments, the compound having the structure:

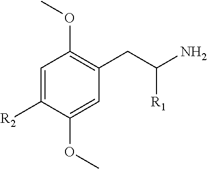

or a pharmaceutically acceptable salt thereof.

In some embodiments, the compound having the structure:

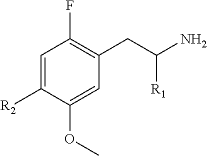

or a pharmaceutically acceptable salt thereof.

In some embodiments, the compound having the structure:

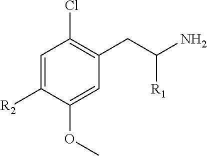

or a pharmaceutically acceptable salt thereof.

In some embodiments, the compound having the structure:

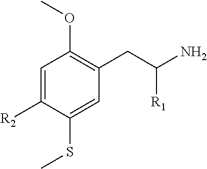

or a pharmaceutically acceptable salt thereof.

In some embodiments, the compound having the structure:

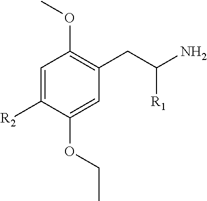

or a pharmaceutically acceptable salt thereof.

In some embodiments, the compound having the structure:

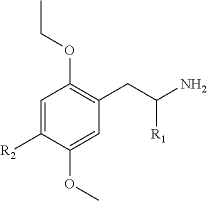

or a pharmaceutically acceptable salt thereof.

In some embodiments, the compound having the structure:

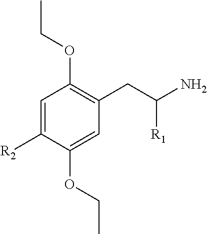

or a pharmaceutically acceptable salt thereof.

In some embodiments, the compound wherein
R₁ is —CH₂CH₃.

In some embodiments, the compound wherein
R₁ is —CH₂CH₂CH₃.

In some embodiments, the compound wherein
R₁ is —CH₂CH═CH₂.

In some embodiments, the compound wherein
R₁ is cyclopropyl.

In some embodiments, the compound wherein
R₂ is H, —CN, —CF₃, -(alkyl), -(alkenyl), -(alkynyl), -(cycloalkyl), -(heterocycle), —S-(aryl) or —Si(CH₃)₃.

In some embodiments, the compound wherein
R₂ is —CN, —CF₃, —CH₃, —CCH, -cyclopropyl, -cyclobutyl, 2-oxetanyl, —S-(phenyl) or —Si(CH₃)₃.

In some embodiments, the compound wherein
R₂ is —CF₃, —CH₂OCH₃ or —CF₂OCH₃.

In some embodiments, the compound wherein
R₂ is —CN or —CF₃.

In some embodiments, the compound wherein
R₁ is —CH₂CH₃, —CH₂CH₂CH₃, —CH₂CH═CH₂ or cyclopropyl; and
R₂ is —CN or —CF₃.

In some embodiments, the compound wherein
R₁ is —CH₂CH₃, and R₂ is —CN or —CF₃.

In some embodiments, the compound wherein
R₁ is —CH₂CH₂CH₃, and R₂ is —CN or —CF₃.

In some embodiments, the compound wherein
R₁ is —CH₂CH═CH₂, and R₂ is —CN or —CF₃.

In some embodiments, the compound wherein
R₁ is cyclopropyl, and R₂ is —CN or —CF₃.

In some embodiments, the compound wherein
R₂ is —CF₃, —CH₂OCH₃, —CF₂CH₃, —CF₂OCH₃ or —SO₂CH₃.

In some embodiments, the compound wherein
R₁ is —CH₂CH₃, —CH₂CH₂CH₃, —CH₂CH═CH₂ or cyclopropyl; and
R₂ is —CF₃, —CH₂OCH₃, —CF₂CH₃, —CF₂OCH₃ or —SO₂CH₃.

In some embodiments, the compound wherein
R₁ is —CH₂CH₃, and R₂ is CF₃, —CH₂OCH₃, —CF₂CH₃, —CF₂OCH₃ or —SO₂CH₃.

In some embodiments, the compound wherein
R₁ is —CH₂CH₂CH₃, and R₂ is CF₃, —CH₂OCH₃, —CF₂CH₃, —CF₂OCH₃ or —SO₂CH₃.

In some embodiments, the compound wherein
R₁ is —CH₂CH═CH₂, and R₂ is CF₃, —CH₂OCH₃, —CF₂CH₃, —CF₂OCH₃ or —SO₂CH₃.

In some embodiments, the compound wherein
R₁ is cyclopropyl, and R₂ is CF₃, —CH₂OCH₃, —CF₂CH₃, —CF₂OCH₃ or —SO₂CH₃.

In some embodiments, the compound wherein
R₁ is —CH₂CH₃,
R₂ is —CF₃, —CH₂OCH₃ or —CF₂OCH₃.

In some embodiments, the compound having the structure:

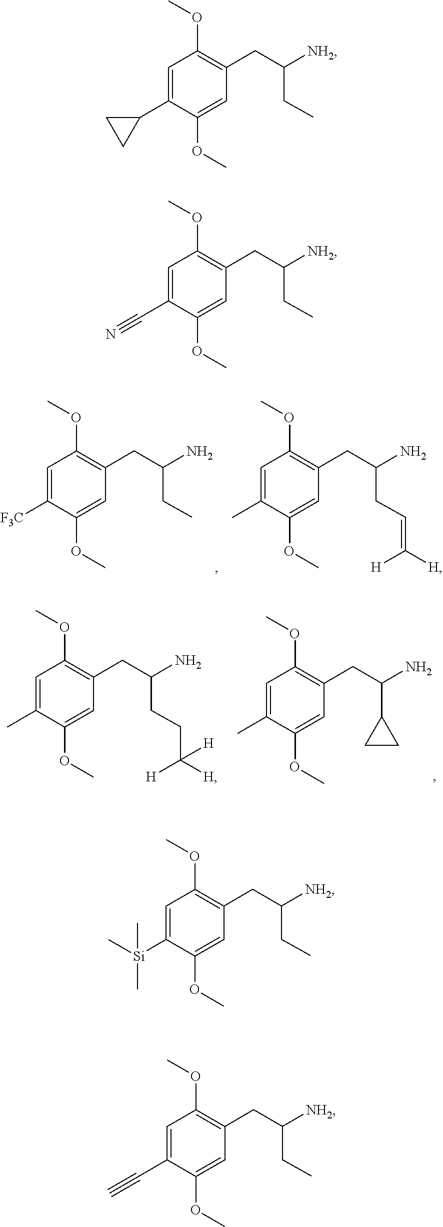

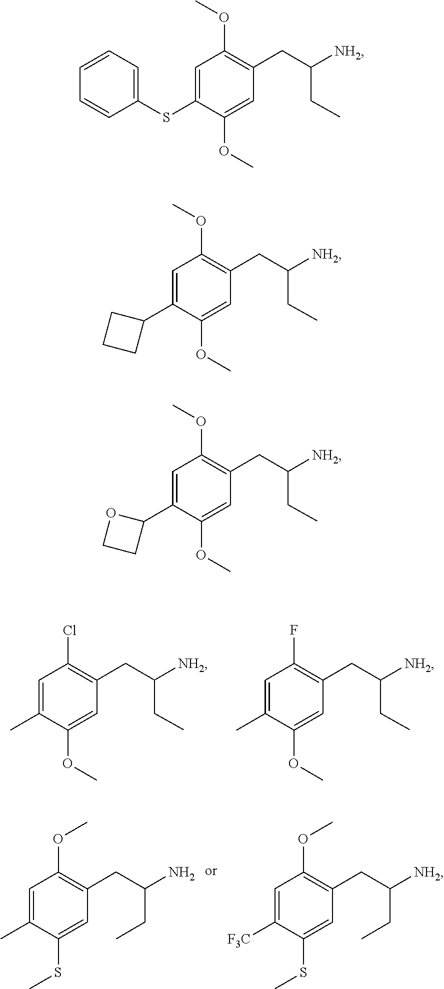

or a pharmaceutically acceptable salt thereof.

In some embodiments, the compound having the structure:

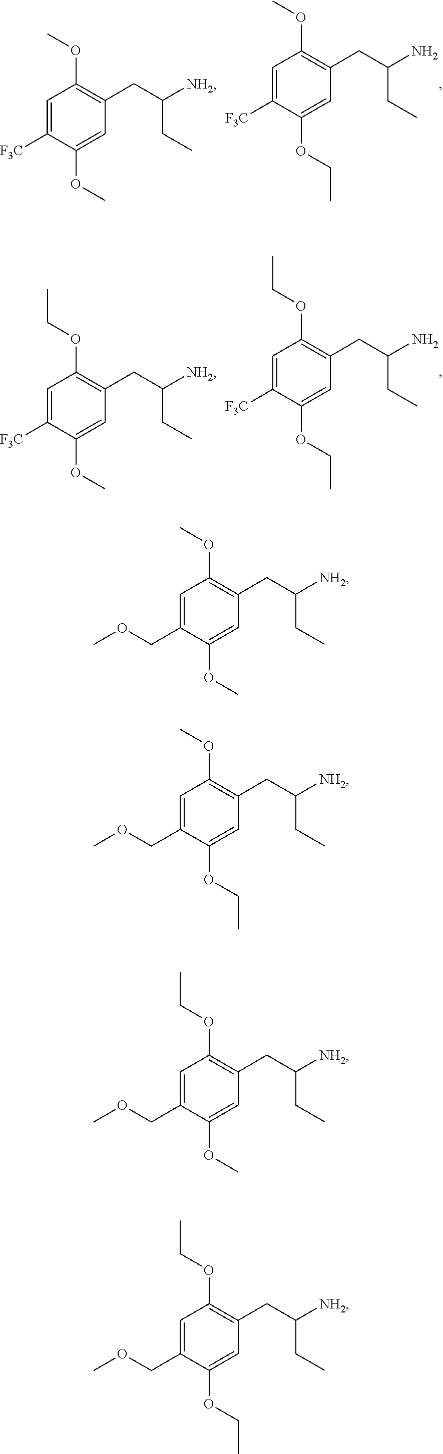

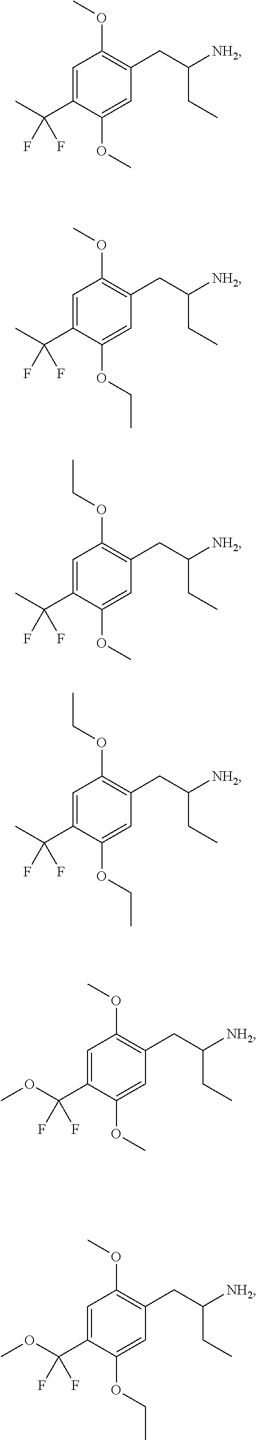

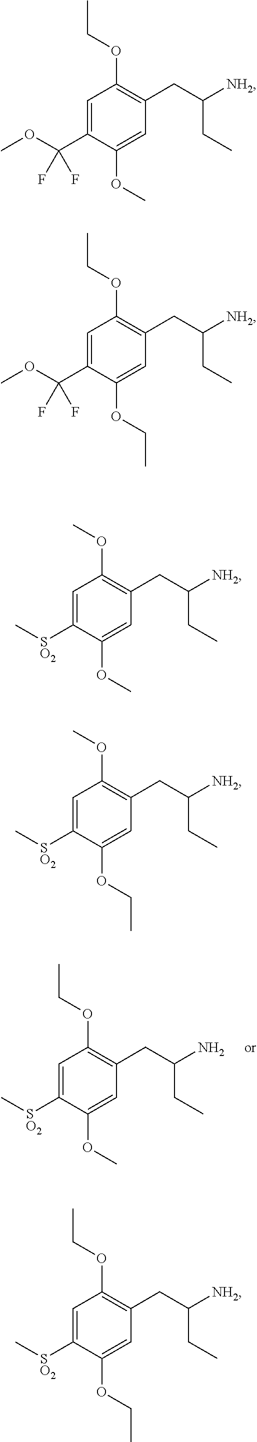

or a pharmaceutically acceptable salt thereof.

In some embodiments, the compound wherein
R₁ is —CH₂CH₃,
R₂ is —CF₃, —CH₂OCH₃ or —CF₂OCH₃,
R₃ is —OCH₃; and
R₄ is —OCH₃.

In some embodiments, the compound having the structure:

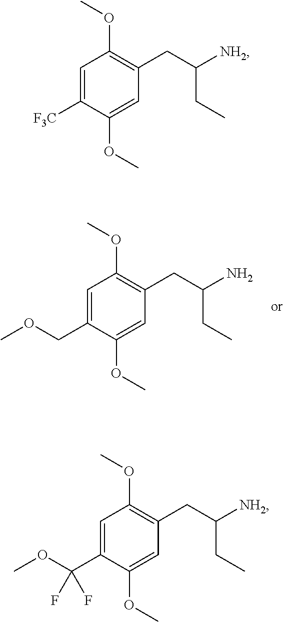

or a pharmaceutically acceptable salt thereof.

In some embodiments, the compound having the structure:

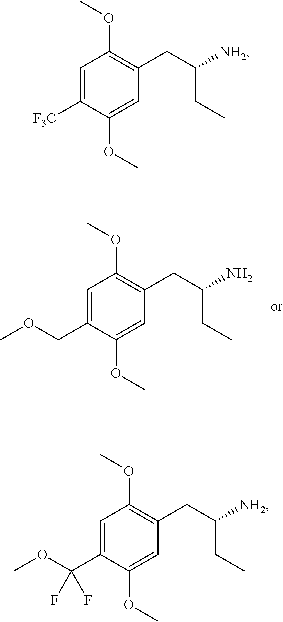

or a pharmaceutically acceptable salt thereof.

The present invention also provides a pharmaceutical composition comprising the compound of the present application and a pharmaceutically acceptable carrier.

The present invention also provides a method of activating 5HT2A receptor comprising contacting the 5HT2A receptor with the compound of the present application.

The present invention also provides a method of selectively activating 5HT2A receptor compared to 5HT2B, comprising selectively contacting the 5HT2A receptor compared to 5HT2B with the compound of the present application.

The present invention also provides a method of treating a subject afflicted with Parkinson's disease comprising administering an effective amount of the compound of the present application or the composition of the present application to the subject so as to treat the Parkinson's disease.

The present invention also provides a method of treating a subject afflicted with dementia or Alzheimer's disease comprising administering an effective amount of the compound of the present application or the composition of the present application to the subject so as to treat the dementia or Alzheimer's disease.

The present invention also provides a method of treating a subject afflicted with attention deficit hyperactivity disorder (ADHD) comprising administering an effective amount of the compound of the present application or the composition of the present application to the subject so as to treat the ADHD.

The present invention also provides a method of treating a subject afflicted with schizophrenia comprising administering an effective amount of the compound of the present application or the composition of the present application to the subject so as to treat the schizophrenia.

The present invention also provides a method of treating a subject afflicted with depression, bipolar disorder, anxiety disorder, obsessive-compulsive disorder (OCD) or stress disorder comprising administering an effective amount of the compound of the present application or the composition of the present application to the subject so as to treat the depression, bipolar disorder, anxiety disorder, obsessive-compulsive disorder (OCD) or stress disorder.

The present invention also provides a method of treating a subject afflicted with a substance use disorder comprising administering an effective amount of the compound of the present application or the composition of the present application to the subject so as to treat the substance use disorder.

In some embodiments of the above methods, wherein the substance use disorder is opioid use disorder, alcohol use disorder or stimulant use disorder including nicotine use disorder.

In some embodiments of the above methods, wherein the substance is an opioid.

In some embodiments of any of the above methods, wherein the opioid is morphine, hydromorphone, oxymorphone, codeine, dihydrocodeine, hydrocodone, oxycodone, nalbuphine, butorphanol, etorphine, dihydroetorphine, levorphanol, metazocine, pentazocine, meptazinol, meperidine (pethidine), buprenorphine, methadone, tramadol, tapentadol, mitragynine, 3-deutero-mitragynine, 7-hydroxymitragynine, 3-deutero-7-hydroxymitragynine, mitragynine pseudoindoxyl or tianeptine.

In some embodiments of any of the above methods, wherein the opioid is fentanyl, sufentanil, alfentanil, furanylfentanyl, 3-methylfentanyl, valerylfentanyl, butyrylfentanyl, β-Hydroxythiofentanyl, acrylfentanyl or carfentanil.

In some embodiments of any of the above methods, wherein the stimulant is cocaine, amphetamine, methamphetamine or cathinone and its derivatives.

In some embodiments of any of the above methods, wherein the stimulant is nicotine.

The present invention also provides a method of treating a subject afflicted with opioid withdrawal symptoms comprising administering an effective amount of the compound of the present application or the composition of the present application to the subject so as to treat the opioid withdrawal symptoms.

In some embodiments of the above methods, wherein a symptom of substance use disorder is opioid withdrawal or mitigation of relapse to opioid use or SUD.

In some embodiments of the above methods, wherein the risk of relapse to the use of opioids, alcohol or stimulants is reduced.

In some embodiments of the above methods, wherein self-administration of an opioid, alcohol or stimulant is reduced.

The present invention also provides a method of treating a subject afflicted with cluster headache comprising administering an effective amount of the compound of the present application or the composition of the present application to the subject so as to treat the cluster headache.

The present invention also provides a method of treating a subject afflicted with diabetic retinopathy, dry eyes, macular degeneration or glaucoma comprising administering an effective amount of the compound of the present application or the composition of the present application to the subject so as to treat the diabetic retinopathy, dry eyes, macular degeneration or glaucoma.

The present invention also provides a method of treating a subject afflicted with catatonia, comprising administering an effective amount of the compound of the present application or the composition of the present application to the subject so as to treat the catatonia.

The present invention also provides a method of enhancing alertness in a subject, comprising administering an effective amount of the compound of the present application or the composition of the present application to the subject so as to enhance alertness.

The present invention also provides a method of enhancing ability to learn in a subject, comprising administering an effective amount of the compound of the present application or the composition of the present application to the subject so as to enhance ability to learn.

In some embodiments of any of the above methods, wherein the effective amount of the compound administered to the subject does not induce a stimulant effect.

In some embodiments of any of the above methods, wherein the effective amount of the compound administered to the subject does not induce a hallucinogenic effect.

In some embodiments of any of the above methods, wherein the effective amount of the compound administered to the subject does not induce a stimulant effect and a hallucinogenic effect.

In some embodiments of any of the above methods, wherein the subject is a mammal.

In some embodiments of the above methods, wherein the mammal is a human.

In some embodiments of any of the above methods, wherein the effective amount of 10-500 mg of the compound is administered to the subject.

The present invention provides a method of activating 5HT2A receptor in a subject comprising administering to a subject an effective amount of a compound having the structure:

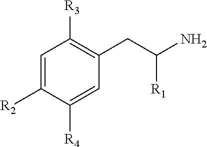

wherein
R₁ is —(C₂-C₁₂ alkyl), -(alkenyl), -(alkynyl), -(cycloalkyl);
R₂ is H, halogen, —NO₂, —CN, —CF₃, —CF₂H, —CH₂OCH₃, —CF₂CH₃, —CF₂OCH₃, -(alkyl), -(alkenyl), -(alkynyl), -(cycloalkyl), -(cycloalkylalkyl), -(heteroalkyl), -(heterocycle), -(heterocycloalkyl), -(aryl), -(heteroaryl), -(hydroxyalkyl), -(haloalkyl), -(alkylaryl), —OH, —O-(alkyl), —O-(alkenyl), —O-(alkynyl), —O-(haloalkyl), —O-(aryl), —O-(heteroaryl), —OCF₃, SH, —S-(alkyl), —S-(alkenyl), —S-(alkynyl), —S-(aryl), —S-(heteroaryl), —NH₂, —NH-(alkyl), —NH-(alkenyl), —NH-(alkynyl) —N-(alkyl)₂, NH-(aryl), —NH-(heteroaryl), —CO₂H, —CO₂-(alkyl), —C(O)—NH₂, —C(O)—NH-(alkyl), —C(O)—NH-(aryl), —SO₂CH₃ or —Si(CH₃) ₃;
R₃ is —OCH₃, —OCH₂CH₃, —F or —Cl; and
R₄ is —OCH₃, —OCH₂CH₃, —SCH₃, —F or —Cl,
   or a pharmaceutically acceptable salt thereof, so as to thereby activate 5HT2A receptor in a subject.

The present invention also provides a method of activating 5HT2A receptor in a subject comprising administering to a subject an effective amount of a compound having the structure:

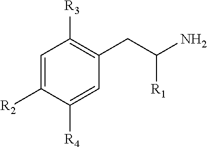

wherein
R₁ is —(C₂-C₁₂ alkyl), -(alkenyl), -(alkynyl), -(cycloalkyl);
R₂ is H, halogen, —NO₂, —CN, —CF₃, —CF₂H, -(alkyl), -(alkenyl), -(alkynyl), -(cycloalkyl), -(cycloalkylalkyl), -(heteroalkyl), -(heterocycle), -(heterocycloalkyl), -(aryl), -(heteroaryl), -(hydroxyalkyl), -(haloalkyl), -(alkylaryl), —OH, —O-(alkyl), —O-(alkenyl), —O-(alkynyl), —O-(haloalkyl), —O-(aryl), —O-(heteroaryl), —OCF₃, SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —N-(alkyl)₂, NH-(aryl), —NH-(heteroaryl), —CO₂H, —CO₂-(alkyl), —C(O)—NH₂, —C(O)—NH-(alkyl), —C(O)—NH-(aryl) or —Si(CH₃)₃;
R₃ is —OCH₃, —F or —Cl; and
R₄ is —OCH₃, —SCH₃, —F or —Cl;
   or a pharmaceutically acceptable salt thereof, so as to thereby activate 5HT2A receptor in a subject.

The present invention provides a method of treating a subject afflicted with Parkinson's disease by activating 5HT2A receptor in a subject.

The present invention provides a method of treating a subject afflicted with dementia or Alzheimer's disease by activating 5HT2A receptor in a subject.

The present invention provides a method of treating a subject afflicted with attention deficit hyperactivity disorder (ADHD) by activating 5HT2A receptor in a subject.

The present invention provides a method of treating a subject afflicted with schizophrenia by activating 5HT2A receptor in a subject.

The present invention provides a method of treating a subject afflicted with depression, bipolar disorder, anxiety disorder, obsessive-compulsive disorder (OCD) or stress disorder by activating 5HT2A receptor in a subject.

The present invention provides a method of treating a subject afflicted with a substance use disorder by activating 5HT2A receptor in a subject.

The present invention provides a method of treating a subject afflicted with opioid use disorder by activating 5HT2A receptor in a subject.

The present invention provides a method of treating a subject afflicted with alcohol use disorder by activating 5HT2A receptor in a subject.

The present invention provides a method of treating a subject afflicted with stimulant use disorder by activating 5HT2A receptor in a subject.

The present invention provides a method of treating a subject afflicted with nicotine use disorder by activating 5HT2A receptor in a subject.

In some embodiments of any of the above methods, wherein the substance is an opioid.

In some embodiments of any of the above methods, wherein the opioid is morphine, hydromorphone, oxymorphone, codeine, dihydrocodeine, hydrocodone, oxycodone, nalbuphine, butorphanol, etorphine, dihydroetorphine, levorphanol, metazocine, pentazocine, meptazinol, meperidine (pethidine), buprenorphine, methadone, tramadol, tapentadol, mitragynine, 3-deutero-mitragynine, 7-hydroxymitragynine, 3-deutero-7-hydroxymitragynine, mitragynine pseudoindoxyl or tianeptine.

In some embodiments of any of the above methods, wherein the opioid is fentanyl, sufentanil, alfentanil, furanylfentanyl, 3-methylfentanyl, valerylfentanyl, butyrylfentanyl, β-Hydroxythiofentanyl, acrylfentanyl or carfentanil.

In some embodiments of any of the above methods, wherein the stimulant is cocaine, amphetamine, methamphetamine or cathinone and its derivatives.

In some embodiments of any of the above methods, wherein the stimulant is nicotine.

The present invention provides a method of treating a subject afflicted with opioid withdrawal symptoms by activating 5HT2A receptor in a subject.

In some embodiments of any of the above methods, wherein a symptom of substance use disorder is opioid withdrawal or mitigation of relapse to opioid use or SUD.

In some embodiments of any of the above methods, wherein the risk of relapse to the use of opioids, alcohol or stimulants is reduced.

In some embodiments of any of the above methods, wherein self-administration of an opioid, alcohol or stimulant is reduced.

The present invention provides a method of treating a subject afflicted with cluster headache by activating 5HT2A receptor in a subject.

The present invention provides a method of treating a subject afflicted with diabetic retinopathy, dry eyes, macular degeneration or glaucoma by activating 5HT2A receptor in a subject.

In some embodiments of any of the above methods, wherein the effective amount of the compound administered to the subject does not induce a stimulant effect.

In some embodiments of any of the above methods, wherein the effective amount of the compound administered to the subject does not induce a hallucinogenic effect.

In some embodiments of any of the above methods, wherein the effective amount of the compound administered to the subject does not induce a stimulant effect and a hallucinogenic effect.

In some embodiments of any of the above methods, wherein the subject is a mammal.

In some embodiments of the above methods, wherein the mammal is a human.

In some embodiments of any of the above methods, wherein an effective amount of 10-500 mg of the compound is administered to the subject.

In some embodiments of the above methods, the compound has the structure:

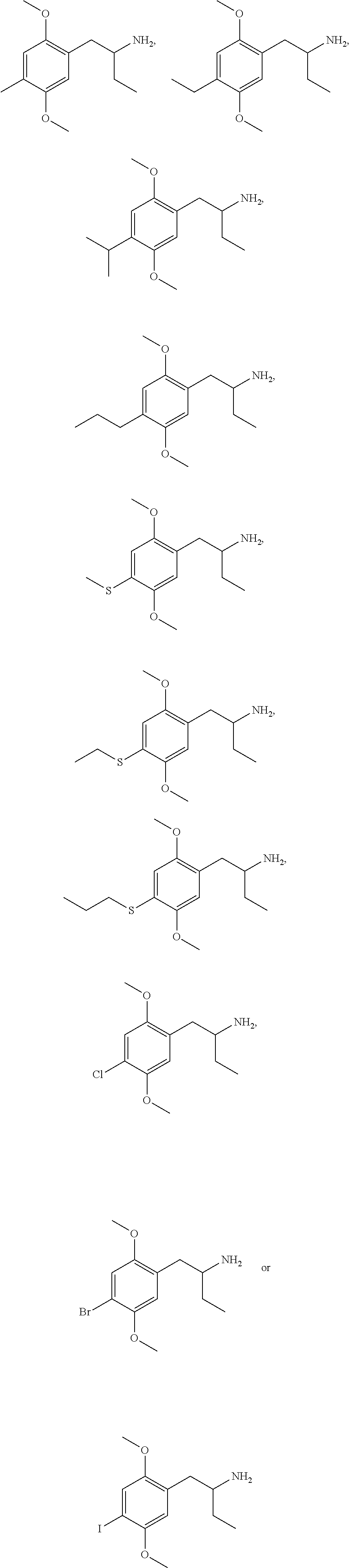

or a pharmaceutically acceptable salt thereof.

In some embodiments of the above methods, the compound has the structure:

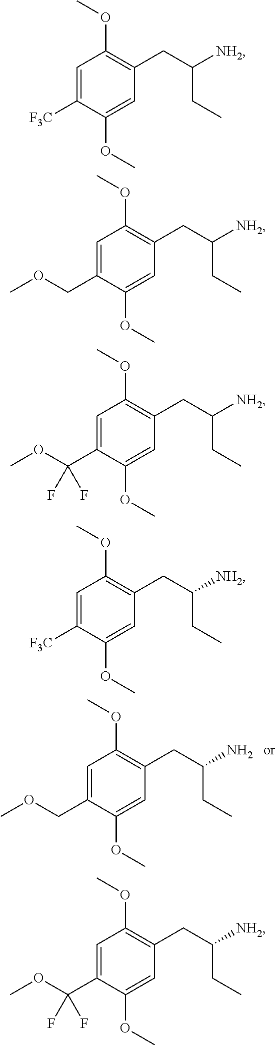

or a pharmaceutically acceptable salt thereof.

Opioid use disorder (OUD) involves, but is not limited to, misuse of opioid medications or use of illicitly obtained opioids. The Diagnostic and Statistical Manual of Mental Disorders, 5th Edition (American Psychiatric Association: Diagnostic and Statistical Manual of Mental Disorders: Diagnostic and Statistical Manual of Mental Disorders, Fifth Edition. Arlington, VA: American Psychiatric Association, 2013), which is hereby incorporated by reference, describes opioid use disorder as a problematic pattern of opioid use leading to problems or distress, with at least two of the following occurring within a 12-month period:
Taking larger amounts or taking drugs over a longer period than intended.
Persistent desire or unsuccessful efforts to cut down or control opioid use.
Spending a great deal of time obtaining or using the opioid or recovering from its effects.
Craving, or a strong desire or urge to use opioids.
Problems fulfilling obligations at work, school, or home.
Continued opioid use despite having recurring social or interpersonal problems.
Giving up or reducing activities because of opioid use.
Using opioids in physically hazardous situations.
Continued opioid use despite ongoing physical or psychological problem likely to have been caused or worsened by opioids.
Tolerance (i.e., need for increased amounts or diminished effect with continued use of the same amount).
Experiencing withdrawal (opioid withdrawal syndrome) or taking opioids (or a closely related substance) to relieve or avoid withdrawal symptoms.

Alcohol use disorder (AUD) involves, but is not limited to, a chronic relapsing brain disease characterized by compulsive alcohol use, loss of control over alcohol intake, and a negative emotional state when not using. The Diagnostic and Statistical Manual of Mental Disorders, 5th Edition describes alcohol use disorder as a problematic pattern of alcohol use leading to problems or distress, with at least two of the following occurring within a 12-month period:
Being unable to limit the amount of alcohol you drink.
Wanting to cut down on how much you drink or making unsuccessful attempts to do so.
Spending a lot of time drinking, getting alcohol, or recovering from alcohol use.
Feeling a strong craving or urge to drink alcohol.
Failing to fulfill major obligations at work, school or home due to repeated alcohol use.
Continuing to drink alcohol even though you know it is causing physical, social, or interpersonal problems.
Giving up or reducing social and work activities and hobbies.
Using alcohol in situations where it is not safe, such as when driving or swimming.
Developing a tolerance to alcohol so you need more to feel its effect, or you have a reduced effect from the same amount.
Experiencing withdrawal symptoms—such as nausea, sweating and shaking—when you do not drink, or drinking to avoid these symptoms.

Stimulant use disorder involves, but is not limited to, a pattern of problematic use of amphetamine, methamphetamine, cocaine, or other stimulants except caffeine or nicotine, leading to at least two of the following problems within a 12-month period:
Taking more stimulants than intended.
Unsuccessful in trying to cut down or control use of stimulants, despite wanting to do so.
Spending excessive amounts of time to activities surrounding stimulant use.
Urges and cravings for stimulants.
Failing in the obligations of home, school, or work.
Carrying on taking stimulants, even though it has led to relationship or social problems.
Giving up or reducing important recreational, social, or work-related activities because of using stimulants.
Using stimulants in a physically hazardous way.
Continuing to use stimulants even while knowing that it is causing or worsening a physical or psychological problem.
Tolerance to stimulants.
Withdrawal from stimulants if you do not take them.

Polydrug use disorder or polysubstance use disorder involves, but is not limited to, dependence on multiple drugs or substances.

The term “5HT2A” refers to the serotonin 2A receptor.

The term “5HT2B” refers to the serotonin 2B receptor.

The term “5HT2A agonist” or “5HT2A receptor agonist” is intended to mean any compound or substance that activates the 5HT2A receptor. The agonist may be a partial, full, super, or biased agonist.

The term “non-hallucinogenic psychedelics” is intended to mean 5HT2A receptor agonists that induce no or limited hallucinogenic effects at pharmacologically and physiologically meaningful engagement of the target receptor.

The term “PTSD” refers to post-traumatic stress disorder.

The term “OCD” refers to obsessive-compulsive disorder.

The term “ADHD” refers to attention deficit hyperactivity disorder.

The term “LSD” refers to lysergic acid diethylamide.

Except where otherwise specified, the structure of a compound of this invention includes an asymmetric carbon atom, it is understood that the compound occurs as a racemate, racemic mixture, and isolated single enantiomer. All such isomeric forms of these compounds are expressly included in this invention. Except where otherwise specified, each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wilen, Pub. John Wiley & Sons, N Y, 1981. For example, the resolution may be carried out by preparative chromatography on a chiral column.

The subject invention is also intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notation of a carbon in structures throughout this application, when used without further notation, are intended to represent all isotopes of carbon, such as ¹²C, ¹³C, or ¹⁴C. Furthermore, any compounds containing ¹³C or ¹⁴C may specifically have the structure of any of the compounds disclosed herein.

It will also be noted that any notation of a hydrogen in structures throughout this application, when used without further notation, are intended to represent all isotopes of hydrogen, such as ¹H, ²H, or ³H. Furthermore, any compounds containing ²H or ³H may specifically have the structure of any of the compounds disclosed herein.

Isotopically-labeled compounds can generally be prepared by conventional techniques known to those skilled in the art using appropriate isotopically-labeled reagents in place of the non-labeled reagents employed.

In the compounds used in the method of the present invention, the substituents may be substituted or unsubstituted, unless specifically defined otherwise.

In the compounds used in the method of the present invention, alkyl, heteroalkyl, monocycle, bicycle, aryl, heteroaryl and heterocycle groups can be further substituted by replacing one or more hydrogen atoms with alternative non-hydrogen groups. These include, but are not limited to, halo, hydroxy, mercapto, amino, carboxy, cyano and carbamoyl.

It is understood that substituents and substitution patterns on the compounds used in the method of the present invention can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure results.

In choosing the compounds used in the method of the present invention, one of ordinary skill in the art will recognize that the various substituents, i.e. R₁, R₂, etc. are to be chosen in conformity with well-known principles of chemical structure connectivity.

As used herein, “alkyl” is intended to include both branched and straight-chain saturated aliphatic hydrocarbon groups having the specified number of carbon atoms. Thus, C₁-Cₙ as in “C₁-Cₙ alkyl” is defined to include groups having 1, 2 . . . . . . , n−1 or n carbons in a linear or branched arrangement, and specifically includes methyl, ethyl, propyl, butyl, pentyl, hexyl, heptyl, isopropyl, isobutyl, sec-butyl and so on. An embodiment can be C₁-C₁₂ alkyl, C₂-C₁₂ alkyl, C₃-C₁₂ alkyl, C₄-C₁₂ alkyl and so on. An embodiment can be C₁-C₈ alkyl, C₂-C₈ alkyl, C₃-C₈ alkyl, C₄-C₈ alkyl and so on. “Alkoxy” represents an alkyl group as described above attached through an oxygen bridge.

The term “alkenyl” refers to a non-aromatic hydrocarbon radical, straight or branched, containing at least 1 carbon to carbon-to-carbon double bond, and up to the maximum possible number of non-aromatic carbon-carbon double bonds may be present. Thus, C₂-Cₙ alkenyl is defined to include groups having 1, 2 . . . , n−1 or n carbons. For example, “C₂-C₆ alkenyl” means an alkenyl radical having 2, 3, 4, 5, or 6 carbon atoms, and at least 1 carbon-carbon double bond, and up to, for example, 3 carbon-carbon double bonds in the case of a C₆ alkenyl, respectively. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated. An embodiment can be C₂-C₁₂ alkenyl or C₂-C₈ alkenyl.

The term “alkynyl” refers to a hydrocarbon radical straight or branched, containing at least 1 carbon-to-carbon triple bond, and up to the maximum possible number of non-aromatic carbon-carbon triple bonds may be present. Thus, C₂-Cₙ alkynyl is defined to include groups having 1, 2 . . . , n−1 or n carbons. For example, “C₂-C₆ alkynyl” means an alkynyl radical having 2 or 3 carbon atoms, and 1 carbon-carbon triple bond, or having 4 or 5 carbon atoms, and up to 2 carbon-carbon triple bonds, or having 6 carbon atoms, and up to 3 carbon-carbon triple bonds. Alkynyl groups include ethynyl, propynyl and butynyl. As described above with respect to alkyl, the straight or branched portion of the alkynyl group may contain triple bonds and may be substituted if a substituted alkynyl group is indicated. An embodiment can be a C₂-Cₙ alkynyl. An embodiment can be C₂-C₁₂ alkynyl or C₃-Cₑ alkynyl.

As used herein, “hydroxyalkyl” includes alkyl groups as described above wherein one or more bonds to hydrogen contained therein are replaced by a bond to an —OH group. In some embodiments, C₁-C₁₂ hydroxyalkyl or C₁-C₆ hydroxyalkyl. C₁-Cₙ as in “C₁-Cₙ alkyl” is defined to include groups having 1, 2, . . . , n−1 or n carbons in a linear or branched arrangement (e.g. C₁-C₂ hydroxyalkyl, C₁-C₃ hydroxyalkyl, C₁-C₄ hydroxyalkyl, C₁-C₅ hydroxyalkyl, or C₁-C₆ hydroxyalkyl) For example, C₁-C₆, as in “C₁-C₆ hydroxyalkyl” is defined to include groups having 1, 2, 3, 4, 5, or 6 carbons in a linear or branched alkyl arrangement wherein a hydrogen contained therein is replaced by a bond to an —OH group.

As used herein, “heteroalkyl” includes both branched and straight-chain saturated aliphatic hydrocarbon groups having the specified number of carbon atoms and at least 1 heteroatom within the chain or branch.

In some embodiments, the haloalkyl is fluoroalkyl. In some embodiments, the fluoroalkyl is —CF₃ or —CH₂F.

As used herein, “monocycle” includes any stable polyatomic carbon ring of up to 10 atoms and may be unsubstituted or substituted. Examples of such non-aromatic monocycle elements include but are not limited to: cyclobutyl, cyclopentyl, cyclohexyl, and cycloheptyl. Examples of such aromatic monocycle elements include but are not limited to: phenyl.

As used herein, “bicycle” includes any stable polyatomic carbon ring of up to 10 atoms that is fused to a polyatomic carbon ring of up to 10 atoms with each ring being independently unsubstituted or substituted. Examples of such non-aromatic bicycle elements include but are not limited to: decahydronaphthalene. Examples of such aromatic bicycle elements include but are not limited to: naphthalene.

As used herein, “aryl” is intended to mean any stable monocyclic, bicyclic or polycyclic carbon ring of up to 10 atoms in each ring, wherein at least one ring is aromatic, and may be unsubstituted or substituted. Examples of such aryl elements include but are not limited to: phenyl, p-toluenyl (4-methylphenyl), naphthyl, tetrahydro-naphthyl, indanyl, phenanthryl, anthryl or acenaphthyl. In cases where the aryl substituent is bicyclic and one ring is non-aromatic, it is understood that attachment is via the aromatic ring.

The term “heteroaryl”, as used herein, represents a stable monocyclic, bicyclic or polycyclic ring of up to 10 atoms in each ring, wherein at least one ring is aromatic and contains from 1 to 4 heteroatoms selected from the group consisting of O, N and S. Bicyclic aromatic heteroaryl groups include phenyl, pyridine, pyrimidine or pyridazine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The term “heterocycle”, “heterocyclyl” or “heterocyclic” refers to a mono- or poly-cyclic ring system which can be saturated or contains one or more degrees of unsaturation and contains one or more heteroatoms. Preferred heteroatoms include N, O, and/or S, including N-oxides, sulfur oxides, and dioxides. Preferably the ring is three to ten-membered and is either saturated or has one or more degrees of unsaturation. The heterocycle may be unsubstituted or substituted, with multiple degrees of substitution being allowed. Such rings may be optionally fused to one or more of another “heterocyclic” ring(s), heteroaryl ring(s), aryl ring(s), or cycloalkyl ring(s). Examples of heterocycles include, but are not limited to, oxetane, tetrahydrofuran, pyran, 1,4-dioxane, 1,3-dioxane, piperidine, piperazine, pyrrolidine, morpholine, thiomorpholine, tetrahydrothiopyran, tetrahydrothiophene, 1,3-oxathiolane, and the like.

As used herein, “heterocycloalkyl” is intended to mean a 3- to 10-membered nonaromatic ring containing from 1 to 4 heteroatoms selected from the group consisting of O, N and S, and includes bicyclic groups.

The term “alkylaryl” refers to alkyl groups as described above wherein one or more bonds to hydrogen contained therein are replaced by a bond to an aryl group as described above. It is understood that an “alkylaryl” group is connected to a core molecule through a bond from the alkyl group and that the aryl group acts as a substituent on the alkyl group. Examples of arylalkyl moieties include, but are not limited to, benzyl (phenylmethyl), p-trifluoromethylbenzyl (4-trifluoromethylphenylmethyl), 1-phenylethyl, 2-phenylethyl, 3-phenylpropyl, 2-phenylpropyl and the like.

As used herein, “cycloalkyl” includes cyclic rings of alkanes of three to eight total carbon atoms, or any number within this range (i.e., cyclopropyl, cyclobutyl, cyclopentyl, cyclohexyl, cycloheptyl or cyclooctyl).

The term “ester” is intended to a mean an organic compound containing the R—O—CO—R′ group.

The term “amide” is intended to a mean an organic compound containing the R—CO—NH—R′ or R—CO—N—R′R″ group.

The term “phenyl” is intended to mean an aromatic six membered ring containing six carbons.

The term “benzyl” is intended to mean a —CH₂R₁ group wherein the R₁ is a phenyl group.

The term “substitution”, “substituted” and “substituent” refers to a functional group as described above in which one or more bonds to a hydrogen atom contained therein are replaced by a bond to non-hydrogen or non-carbon atoms, provided that normal valencies are maintained and that the substitution results in a stable compound. Substituted groups also include groups in which one or more bonds to a carbon(s) or hydrogen(s) atom are replaced by one or more bonds, including double or triple bonds, to a heteroatom. Examples of substituent groups include the functional groups described above, and halogens (i.e., F, Cl, Br, and I); alkyl groups, such as methyl, ethyl, n-propyl, isopropyl, n-butyl, tert-butyl, and trifluoromethyl; hydroxyl; alkoxy groups, such as methoxy, ethoxy, n-propoxy, and isopropoxy; aryloxy groups, such as phenoxy; arylalkyloxy, such as benzyloxy (phenylmethoxy) and p-trifluoromethylbenzyloxy (4-trifluoromethylphenylmethoxy); heteroaryloxy groups; sulfonyl groups, such as trifluoromethanesulfonyl, methanesulfonyl, and p-toluenesulfonyl; nitro, nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

The compounds used in the method of the present invention may be prepared by techniques well known in organic synthesis and familiar to a practitioner ordinarily skilled in the art. However, these may not be the only means by which to synthesize or obtain the desired compounds.

The compounds used in the method of the present invention may be prepared by techniques described in Vogel's Textbook of Practical Organic Chemistry, A. I. Vogel, A. R. Tatchell, B. S. Furnis, A. J. Hannaford, P. W. G. Smith, (Prentice Hall) 5^{th} Edition (1996), March's Advanced Organic Chemistry: Reactions, Mechanisms, and Structure, Michael B. Smith, Jerry March, (Wiley-Interscience) 5^{th} Edition (2007), and references therein, which are incorporated by reference herein. However, these may not be the only means by which to synthesize or obtain the desired compounds.

The various R groups attached to the aromatic rings of the compounds disclosed herein may be added to the rings by standard procedures, for example those set forth in Advanced Organic Chemistry: Part B: Reactions and Synthesis, Francis Carey and Richard Sundberg, (Springer) 5th ed. Edition. (2007), the content of which is hereby incorporated by reference.

Another aspect of the invention comprises a compound used in the method of the present invention as a pharmaceutical composition.

As used herein, the term “pharmaceutically active agent” means any substance or compound suitable for administration to a subject and furnishes biological activity or other direct effect in the treatment, cure, mitigation, diagnosis, or prevention of disease, or affects the structure or any function of the subject. Pharmaceutically active agents include, but are not limited to, substances and compounds described in the Physicians' Desk Reference (PDR Network, LLC; 64th edition; Nov. 15, 2009) and “Approved Drug Products with Therapeutic Equivalence Evaluations” (U.S. Department Of Health And Human Services, 30^{th} edition, 2010), which are hereby incorporated by reference. Pharmaceutically active agents which have pendant carboxylic acid groups may be modified in accordance with the present invention using standard esterification reactions and methods readily available and known to those having ordinary skill in the art of chemical synthesis. Where a pharmaceutically active agent does not possess a carboxylic acid group, the ordinarily skilled artisan will be able to design and incorporate a carboxylic acid group into the pharmaceutically active agent where esterification may subsequently be carried out so long as the modification does not interfere with the pharmaceutically active agent's biological activity or effect.

The compounds used in the method of the present invention may be in a salt form. As used herein, a “salt” is a salt of the instant compounds which has been modified by making acid or base salts of the compounds. In the case of compounds used to treat an infection or disease caused by a pathogen, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

As used herein, “treating” means preventing, slowing, halting, or reversing the progression of a disease or infection. Treating may also mean improving one or more symptoms of a disease or infection.

The compounds used in the method of the present invention may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection, topical application, or other methods, into or onto a site of infection, all using dosage forms well known to those of ordinary skill in the pharmaceutical arts.

The compounds used in the method of the present invention can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds used in the method of the present invention may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines. The compounds may be administered as components of tissue-targeted emulsions.

The compounds used in the method of the present invention may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The compounds used in the method of the present invention may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials to make them compatible with the type of injection or delivery system chosen.

Each embodiment disclosed herein is contemplated as being applicable to each of the other disclosed embodiments. Thus, all combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### Experimental Details

General Considerations. Reagents and solvents were obtained from commercial sources and were used without further purification unless otherwise stated. Reactions were monitored by TLC using solvent mixtures appropriate to each reaction. Column chromatography was performed on silica gel (40-63 μm). For compounds containing a basic nitrogen, Et₃N was often used in the mobile phase to provide better resolution when using silica gel chromatography. In these cases, TLC plates were pre-soaked in the Et₃N-containing solvent and then allowed to dry briefly before use in analysis, such that an accurate representation of R_{f} was obtained. For preparative TLC, glass plates coated with a 1 mm silica layer were used. Nuclear magnetic resonance spectra were recorded on Bruker 400 or 500 MHz instruments, as indicated. Chemical shifts are reported as δ values in ppm referenced to CDCl₃ (¹H NMR=7.26 and ¹³C NMR=77.16) or methanol-d₄ (¹H NMR=3.31 and ¹³C NMR=49.00). Multiplicity is indicated as follows: s (singlet); d (doublet); t (triplet); dd (doublet of doublets); td (triplet of doublets); dt (doublet of triplets); dq (doublet of quartets); ddd (doublet of doublet of doublets); ddt (doublet of doublet of triplets); m (multiplet); br (broad). Low-resolution mass spectra were recorded on an Advion quadrupole instrument (ionization mode: APCI+ or ESI+) or on GC-MS (ionization mode: EI).

### Example 1. General Procedure A: Preparation of Nitrostyrenes

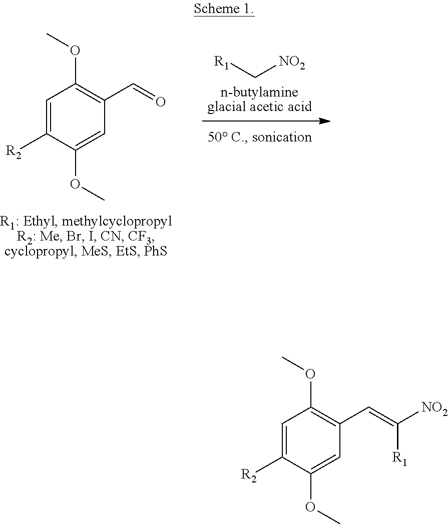

General Procedure A: Preparation of Nitrostyrenes

Benzaldehyde (1 eq) was added to a reaction tube with a magnetic stir bar followed by glacial acetic acid (5.5 eq) and nitropropane (1.5 eq). N-butylamine (2 eq) was then added dropwise with stirring. Reaction mixture was sealed with septum and placed in a sonicating water bath for 16 hours, then diluted with 3× the volume of toluene as the reaction mixture. This solution was transferred immediately to a short silica gel column and purified using 100% toluene as eluent.

### Example 2. General Procedure B: Preparation of Butanamine Products Using Lithium Aluminum Hydride

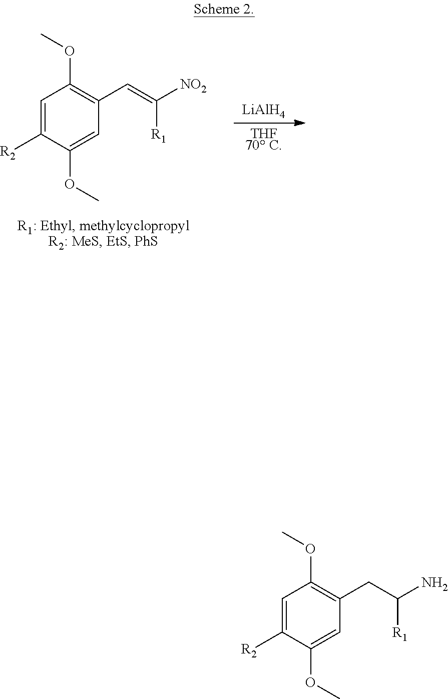

General Procedure B: Preparation of Butanamine Products Using Lithium Aluminum Hydride

Per 1 mmol of nitrostyrene: A solution of 1 mmol of appropriate nitrostyrene in 2 mL of THF was added dropwise to a suspension of 7 mmol of LiAlH₄ in 3.5 mL of THF while stirring under an argon atmosphere. The reaction mixture was heated 18 hours at 70° C., cooled to 0° C. Reaction was carefully quenched with isopropanol (200 uL), water (200 uL), aqueous NaOH solution (200 uL, 15% NaOH), and finally more water (600 uL) then stirred vigorously 30 minutes at room temperature. The suspension was vacuum filtered over celite pad and filter cake washed 3× with ethyl acetate. Solvent was removed under reduced pressure, and the crude product was purified over with flash chromatography (9:1 ethyl acetate/methanol+2% triethylamine).

### Example 3. General Procedure C: Preparation of Butanamine Products Using Alane (Aluminum Hydride)

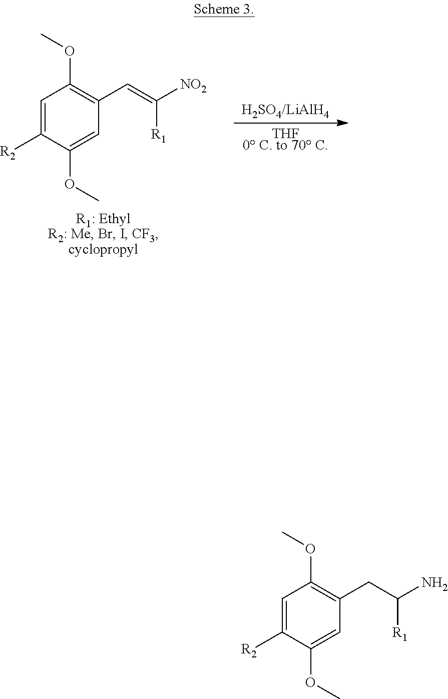

General Procedure C: Preparation of Butanamine Products Using Alane (Aluminum Hydride)

Per 1 mmol of nitrostyrene: Under argon atmosphere, 7 ml of anhydrous THF was added to oven dried scintillation vial containing a stir bar followed by lithium aluminum hydride (5 mmol) rapidly added in 3 portions. Reaction mixture was then cooled with ice water bath 5 minutes before adding sulfuric acid (2.5 mmol, approx. normality of 36) diluted in 2 mL dry THF in slow, dropwise fashion. This mixture was allowed to stir for 20 minutes in ice bath. Appropriate nitrosyrene was dissolved in 3 ml of dry THF and added dropwise (followed by 2×1 ml washes dry THF). The reaction mixture was then heated to 65° C. for 2 hours and then cooled to 0° C. with ice bath. Reaction was diluted with THF (3 mL), isopropanol was added dropwise (200 uL), water was added dropwise (200 uL), aqueous NaOH solution added dropwise (200 uL, 15% NaOH), and more water (600 uL) added dropwise and stirred vigorously 30 minutes at room temperature. The suspension was vacuum filtered over celite pad and filter cake washed 3× with ethyl acetate. Solvent was removed under reduced pressure, and the crude product was purified over with flash chromatography (9:1 ethyl acetate/methanol+2% triethylamine).

### Example 4. General Procedure D: Preparation of 4-alkylthiobenzaldehydes

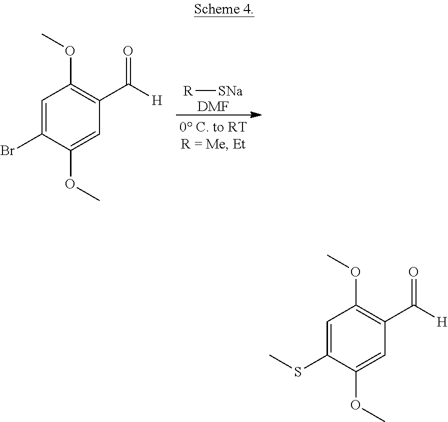

General Procedure D: Preparation of 4-Alkylthiobenzaldehydes

11 mmol of appropriate sodium n-alkanethiolate was added to 25 mL of anhydrous DMF under argon in one portion. This suspension was cooled in ice-bath for 10 min, then 11 mmol of 4-bromo-2,5-dimethoxybenzaldehyde was added in one portion. Reaction mixture was allowed to reach room temperature naturally and to stir for 16 hrs, then it was poured into 400 ml water, off-white precipitate was filtered and allowed to air-dry.

### Example 5. Preparation of 2,5-dimethoxy-4-(trifluoromethyl)benzaldehyde 1

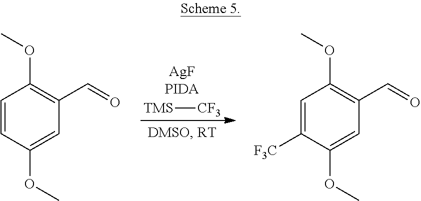 [SMILES: COc1ccc(OC)c(C=O)c1 ; C[Si](C)(C)C(F)(F)F ; COc1cc(C(F)(F)F)c(OC)cc1C=O]

2,5-dimethoxy-4-(trifluoromethyl) benzaldehyde 1 An oven dried 100 mL flask under argon was charged with 2,5-dimethoxybenzaldehyde (15 mmol), (diacetoxyiodo) benzene (30 mmol), trimethyl(trifluoromethyl)silane (30 mmol) and 50 mL anhydrous DMSO. This mixture was stirred at room temperature for 5 minutes, then AgF (3.75 mmol) was slowly added portion-wise to the stirring mixture and was stirred under argon 20 hours. The reaction was quenched by pouring contents of flask into 300 ml water, and extracted 3×100 ml toluene. Pooled organic extracts were washed with brine and dried over MgSO₄. Following removal of solvent under reduced pressure, crude material was purified with flash chromatography using silica gel and 1:3 hexanes/DCM yielding 536 mg of product (15% yield).

¹H NMR (500 MHz, Chloroform-d) δ 10.50 (s, 1H), 7.47 (s, 1H), 7.25 (s, 1H), 3.97 (s, 3H), 3.93 (s, 3H). ¹³C NMR (126 MHz, Chloroform-d) 188.81, 155.31, 151.35, 127.30(m), 123.87, 111.44(m), 110.90, 56.54, 56.37.

### Example 6. Preparation of tert-butyl (1-(4-bromo-2,5-dimethoxyphenyl)butan-2-yl)carbamate 2

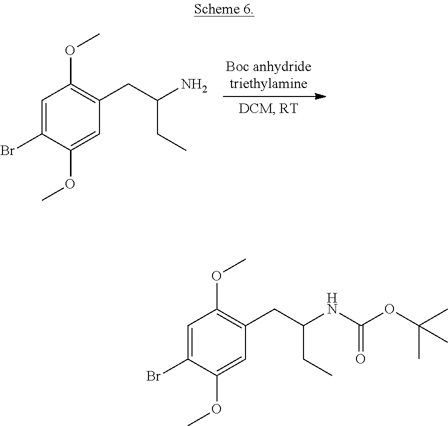 [SMILES: CCC(N)Cc1cc(OC)c(Br)cc1OC ; CCC(Cc1cc(OC)c(Br)cc1OC)NC(=O)OC(C)(C)C]

### tert-butyl (1-(4-bromo-2,5-dimethoxyphenyl) butan-2-yl) carbamate 2

Di-tert-butyl dicarbonate (630 mg, 2.9 mmol, 1.6 eq) was added to a 20 ml scintillation vial followed by 1-(4-bromo-2,5-dimethoxyphenyl)butan-2-amine 21 (520 mg, 1.8 mmol, 1 eq), 10 mL DCM and while stirring, 400 uL (2.9 mmol, 1.6 eq) triethylamine was added dropwise and stirred at room temperature 16 hours. The reaction mixture was diluted with 10 mL DCM, then poured into saturated NH₄Cl (70 mL), washed with NaHCO₃ (70 mL), and brine (70 mL). DCM solution was dried over Na₂SO₄, dried and purified with flash chromatography using silica gel and 1:2 ethyl acetate/hexanes, yielding 400 mg of product (89% yield).

¹H NMR (400 MHz, Chloroform-d) δ 7.02 (s, 1H), 6.74 (s, 1H), 4.47 (s, 1H), 3.84 (s, 3H), 3.78 (s, 3H), 3.75 (s, 1H), 2.74 (m, 2H), 1.60-1.55 (m, 1H), 1.36 (s, 9H), 0.94 (t, J=7.4 Hz, 3H). ¹³C NMR (101 MHz, Chloroform-d) δ 152.14, 149.88, 127.47, 115.73, 56.93, 56.07, 28.36, 27.43, 10.37.

### Example 7. Preparation of tert-butyl (1-(4-cyano-2,5-dimethoxyphenyl)butan-2-yl)carbamate 3

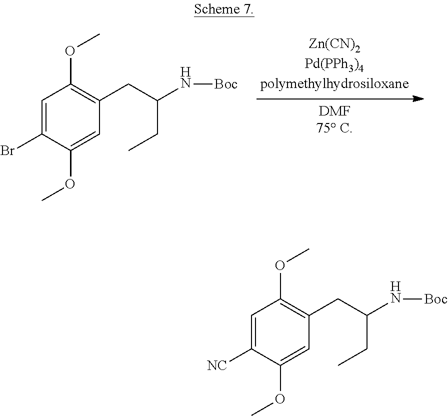 [SMILES: CCC(Cc1cc(OC)c(Br)cc1OC)NC(=O)OC(C)(C)C ; CCC(Cc1cc(OC)c(C#N)cc1OC)NC(=O)OC(C)(C)C]

### tert-butyl (1-(4-cyano-2,5-dimethoxyphenyl) butan-2-yl) carbamate 3

Polymethylhydrosiloxane (8 mg) was added to a 20 ml oven-dried scintillation vial, followed by tert-butyl (1-(4-bromo-2,5-dimethoxyphenyl)butan-2-yl)carbamate 2 (85 mg, 0.22 mmol, 1 eq), followed by zinc cyanide (20 mg, 0.17 mmol, 0.8 eq) and tetrakis(triphenylphosphine)palladium (25 mg, 0.22 mmol, 0.1 eq), then sealed with septum and vacuum/argon cycled 3 times. 5 ml anhydrous DMF was then added and a stream of argon was passed through the suspension with a needle while stirring vigorously for 15 minutes. The reaction was then heated to 75° C. for 16 hours. The reaction mixture was cooled, filtered over a pad of celite, which was rinsed with ethyl acetate (60 mL). The filtrate was washed with 100 ml saturated NaHCO₃, followed by 100 ml dionized water and finally with 100 ml brine. Organic fraction was dried over Na₂SO₄ and the concentrated crude material was subjected to flash chromatography using silica gel and 1:2 ethyl acetate/hexanes, yielding 53 mg of product (72% yield).

¹H NMR (500 MHz, Chloroform-d) δ 6.95 (s, 1H), 6.82 (s, 1H), 3.88 (s, 3H), 3.80 (s, 3H), 2.82 (dd, J=13.9, 5.2 Hz, 1H), 2.72 (dd, J=13.4, 9.5 Hz, 1H), 1.61-1.50 (m, 2H), 1.43 (dd, J=13.7, 6.5 Hz, 1H), 1.35 (s, 9H), 0.96 (t, J=7.4 Hz, 3H). ¹³C NMR (126 MHz, Chloroform-d) δ 155.70, 151.43, 116.79, 114.28, 114.07, 56.42, 55.99, 52.25, 35.65, 28.33, 28.25, 10.41.

### Example 8. Preparation of 4-(2-aminobutyl)-2,5-dimethoxybenzonitrile trifluoroacetate 4

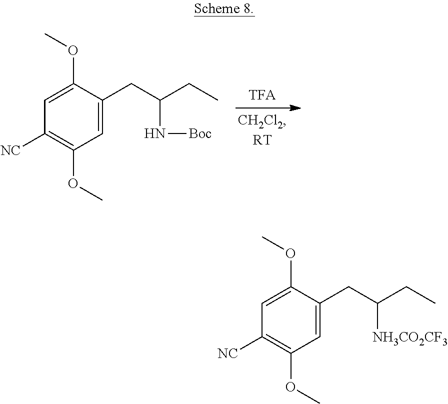

### 4-(2-aminobutyl)-2,5-dimethoxybenzonitrile trifluoroacetate 4

Tert-butyl (1-(4-cyano-2,5-dimethoxyphenyl)butan-2-yl)carbamate (48 mg, 143 umol, 1 eq) was dissolved in 2 ml dry DCM and 66 uL (861 umol, 6 eq) trifluoroacetic acid was added to the stirred solution. The solution was stirred overnight at room temperature before liquids were completely removed by rotary evaporation. This crude material was redissolved in 1 mL water and methanol mixture (3:7) and eluted through C18 cartridge (Thermo-Scientific 50 mg Hypersep column). Solvent was removed under reduced pressure to give 42 mg solid white trifluoroacetate salt product (72% yield).

1H NMR (500 MHz, DMSO-d6) δ 7.86 (s, 2H), 7.36 (s, 1H), 7.13 (s, 1H), 3.87 (s, 3H), 3.79 (s, 3H), 3.36-3.29 (m, 1H), 2.92 (dd, J=13.6, 6.6 Hz, 1H), 2.83 (dd, J=13.6, 7.2 Hz, 1H), 1.57-1.48 (m, 2H), 0.93 (t, J=7.5 Hz, 3H). 13C NMR (126 MHz, DMSO-d6) δ 155.63, 151.68, 132.91, 116.91, 115.85, 115.42, 99.09, 56.93, 56.75, 52.16, 33.60, 25.63, 9.81.

### Example 9. Preparation of 4-cyclopropyl-2,5-dimethoxybenzaldehyde 5

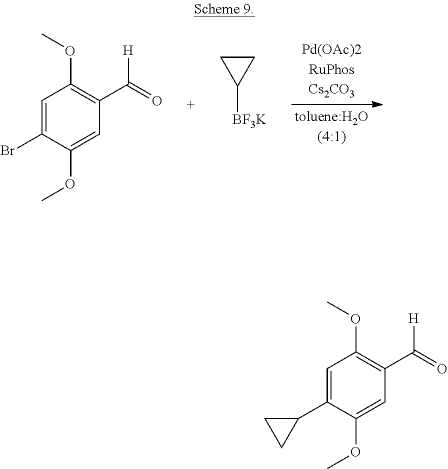

### 4-cyclopropyl-2,5-dimethoxybenzaldehyde 5

4-Bromo-2,5-dimethoxybenzaldehyde (1.2 grams, 4.9 mmol, 1 eq), appropriate potassium cyclobutyl trifluoroborate salt (797 mg, 5.4 mmol, 1.1eq), RuPhos (91 mg, 0.195 mmol, 0.04 eq), palladium acetate (22 mg, 0.098 mmol, 0.02 eq), and cesium carbonate (479 mg, 14.7 mmol, 3 eq) was added to oven dried vial equipped with stir-bar and sealed with septum. The mixture was vacuum/argon cycled 3 times and toluene (11 ml) and water (4 ml) were added. Reaction mixture was stirred vigorously at 95° C. for 40 hours. The reaction mixture was then cooled, filtered over celite and rinsed with ethyl acetate. Filtrate was washed with saturated NH₄Cl solution followed by brine and organic fraction was dried over Na₂SO₄ and concentrated under reduced pressure. Crude material was subjected to flash chromatography using silica gel and hexane/DCM gradient to yield 879 mg of off-white solid (87% yield).

¹H NMR (500 MHz, Chloroform-d) δ 10.37 (s, 1H), 7.26 (s, 1H), 6.40 (s, 1H), 3.85 (d, J=2.9 Hz, 6H), 2.28 (tt, J=8.5, 5.3 Hz, 1H), 1.09-1.02 (m, 2H), 0.78-0.71 (m, 2H). ¹³C NMR (101 MHz, Chloroform-d) δ 189.15, 157.28, 152.55, 142.48, 122.43, 108.39, 108.23, 56.27, 56.21, 10.50, 9.46.

### Example 10. Preparation of 4-iodo-2,5-dimethoxybenzaldehyde 6

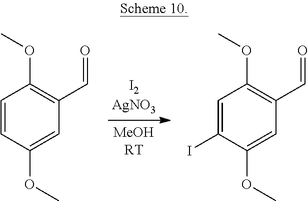

### 4-iodo-2,5-dimethoxybenzaldehyde 6

A mixture of 2,5-dimethoxybenzaldehyde (5.1 g, 31 mmol, 1 eq), silver nitrate (5.21 g, 31 mmol, 1 eq), and iodine (8.1 g, 31.9 mmol, 1.04 eq) in 125 mL of methanol was stirred under argon overnight. The yellow precipitate was filtered and washed with cool methanol. The remaining iodine was reduced with saturated sodium bisulfite solution by addition until the disappearance of yellow color. The solvent was removed on a rotary evaporator and the residue recrystallized from 95% ethanol to yield 8.3 grams of fluffy beige solid (93% yield).

¹H NMR (500 MHz, Chloroform-d) δ 10.40 (s, 1H), 7.47 (s, 1H), 7.22 (s, 1H), 3.90 (s, 3H), 3.87 (s, 3H).

### Example 11. Preparation of 2,5-dimethoxy-4-(phenylthio)benzaldehyde 7

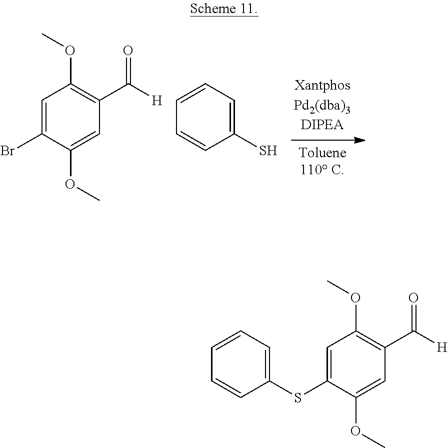

### 2,5-dimethoxy-4-(phenylthio)benzaldehyde 7

To an oven-dried flask was added 4-bromo-2,5-dimethoxy-benzaldehyde (1.5 g, 6.1 mmol, 1 eq), Pd₂(dba)₃ (560 mg, 612 umol, 0.10 eq), Xantphos (354 mg, 612 umol, 0.10 eq), followed by toluene (20 mL), benzenethiol (1.25 mL, 12.24 mmol, 2 eq) and diisopropylethylamine (7.9 g, 10.6 mL, 10 eq). The solution was stirred under argon at 110° C. for 5 hrs. After cooling, the reaction mixture was filtered over celite and rinsed with ethyl acetate and concentrated under reduced pressure. The crude material was subjected to flash chromatography using silica gel and hexane/DCM gradient to yield 1.35 grams of off-white solid (80% yield).

¹H NMR (500 MHz, Chloroform-d) δ 10.31 (s, 1H), 7.61-7.40 (m, 5H), 7.25-7.13 (m, 2H), 3.92 (s, 3H), 3.55 (s, 3H).

### Example 12. Preparation of 2,5-dimethoxy-4-propylbenzaldehyde 8

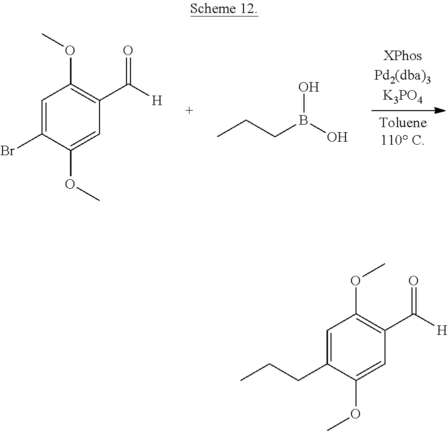

### 2,5-dimethoxy-4-propylbenzaldehyde 8

To an oven-dried flask was added 4-bromo-2,5-dimethoxy-benzaldehyde (7.5 g, 30.6 mmol, 1 eq), Xphos (4.38, 9.18 mmol, 0.30 eq), Pd₂(dba)₃ (2.8 g, 3.06 mmol, 0.10 eq), followed by toluene (300 mL), propyl-boronic acid (4.04 g, 45.9 mmol, 1.5 eq) and K₃PO₄ (19.49 g, 91.81 mmol, 3 eq). The solution was stirred under argon at 110° C. for 18 hrs. After cooling, the reaction mixture was filtered over celite and rinsed with ethyl acetate and concentrated under reduced pressure. The crude material was subjected to flash chromatography using silica gel and ether/hexane gradient to yield 2.55 grams of pale yellow oil (40% yield).

¹H NMR (300 MHz, CDCl₃) δ 10.47-10.30 (m, 1H), 7.26 (s, 1H), 6.78 (s, 1H), 3.87 (s, 3H), 3.80 (s, 3H), 2.72-2.53 (m, 2H), 1.74-1.49 (m, 2H), 1.05-0.87 (m, 3H). ¹³C NMR (300 MHz, CDCl₃) δ 189.17, 156.69, 151.77, 140.92, 122.89, 113.91, 108.08, 56.16, 55.76, 33.07, 22.72, 13.99.

### Example 13. Preparation of 5-ethoxy-2-methoxybenzaldehyde 9

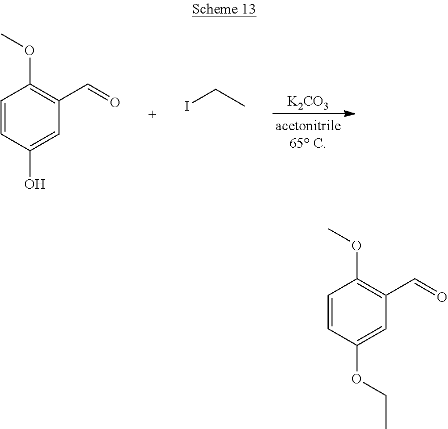

### 5-ethoxy-2-methoxybenzaldehyde 9

5-hydroxy-2-methoxybenzaldehyde (1.5 g, 9.86 mmol, 1 eq) and anhydrous K₂CO₃ (2.73 g, 19.72 mmol, 2 eq), were added to acetonitrile (15 mL) in a 50-mL flask. The reaction mixture was heated gently at 65° C. for 30 min, iodoethane (6.34 mL, 78.87 mmol, 8 eq) was added dropwise, and was heated at 65° C. for 16 hr. After completion of the reaction, the reaction mixture concentrated partitioned between toluene and water, organics washed with brine. The crude material was subjected to flash chromatography using silica gel and hexane/DCM gradient to yield 1.27 grams of amber oil (71% yield).

### 5-ethoxy-2-methoxy-4-(trifluoromethyl) benzaldehyde 10

Prepared in the same fashion as compound 1 using 5-ethoxy-2-methoxybenzaldehyde 9 as starting material with a yield of 12%

¹H NMR (300 MHz, CDCl₃) δ 10.47 (s, 1H), 7.43 (s, 1H), 7.22 (s, 1H), 4.13 (q, J=7.0 Hz, 2H), 3.94 (s, 3H), 1.42 (t, J=9.7, 4.2 Hz, 3H).

### Example 14. Preparation of (4-bromo-2,5-dimethoxyphenyl)methanol

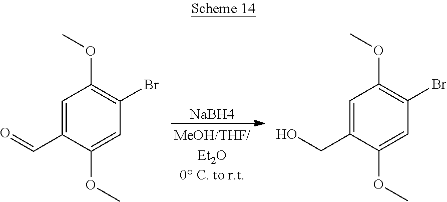

### (4-bromo-2,5-dimethoxyphenyl)methanol

To an oven-dried flask was added 4-bromo-2,5-dimethoxybenzaldehyde (3.2 g 13 mmol, 1 eq), 40 ml MeOH, 10 mL diethyl ether, 10 ml THF. Afer cooling with ice water bath, sodium borohydride (553 mg, 14.6 mmol, 1.12 eq) was added in 2 portions spaced 5 min. Reaction allowed to reach rt naturally and stirred 16 hr. Reaction was poured into 10% HCl and extracted with DCM. Dried over magnesium sulfate, concentrated and used without further purification with yield of 99%.

### Example 15. Preparation of 1-bromo-2,5-dimethoxy-4-(methoxymethyl) benzene

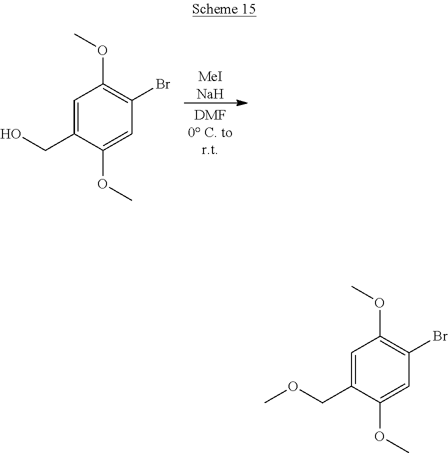

### 1-bromo-2,5-dimethoxy-4-(methoxymethyl)benzene 11

A suspension of (4-bromo-2,5-dimethoxyphenyl)methanol (3.22 g; 13 mmol) in anhydrous dimethylformamide (90 mL) was cooled to 0° C. Then, NaH (60% dispersion in mineral oil; 1.2 mg; 30 mmol; 2.3 eq) was added. The reaction mixture was stirred for 1 h at room temperature. Then methyl iodide (4.46 ml, 71.7 mmol; 5.5 eq) was added and the stirring was continued for further 3 h. The mixture was added to water (250 mL) and extracted with dichloromethane (3×50 mL). The organic layer was dried over anhydrous Na₂SO₄, filtered, and evaporated in vacuo. The crude material was eluted through a silica gel pad with DCM to give a crude solid that was used without further purification with yield of 62%.

### Example 16. Preparation of 2,5-dimethoxy-4-(methoxymethyl)benzaldehyde 12

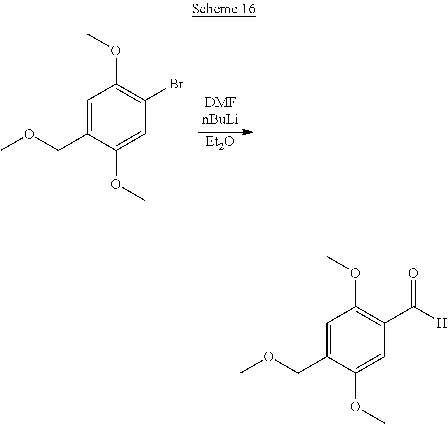

### 2,5-dimethoxy-4-(methoxymethyl) benzaldehyde 12

To a solution of 1-bromo-2,5-dimethoxy-4-(methoxymethyl)benzene (2.07 g, 7.93 mmol, 1 eq) in anhydrous diethyl ether (125 mL) at 0° C. was added n-butyllithium (2M cyclohexanes, 4.44 ml, 8.88 mmol, 1.12 eq) dropwise over five minutes. The solution was stirred at 0° C. for 10 minutes, and DMF (1.84 ml, 23.78 mmol, 3 eq) was added. The reaction was stirred at 0° C. for 5 min, rt 10 min, and heated to 40° C. for 10 min. The reaction was poured into 10% HCl, was extracted with DCM, and was dried over Mg₂SO₄, filtered, and concentrated to a yellow solid. Purification by silica gel chromatography provided product in 49% yield.

1H NMR (400 MHz, CDCl₃) δ 10.43 (s, 1H), 7.28 (s, 1H), 7.12 (s, 1H), 4.52 (d, J=0.8 Hz, 2H), 3.92 (s, 3 Hz), 3.83 (s, 3H), 3.49 (s, 3H)

### Example 17. Preparation of Nitrostyrenes

### (E)-1,4-dimethoxy-2-methyl-5-(2-nitrobut-1-en-1-yl)benzene 13

Prepared according to general procedure A using 2,5-dimethoxy-4-methylbenzaldehyde and nitropropane with yield of 80%.

¹H NMR (400 MHz, Chloroform-d) δ 8.23 (s, 1H), 6.80-6.74 (m, 2H), 3.82 (d, J=8.3 Hz, 6H), 2.85 (q, J=7.4 Hz, 2H), 2.27 (s, 3H), 1.28 (t, J=7.4 Hz, 3H).

### (E)-1-bromo-2,5-dimethoxy-4-(2-nitrobut-1-en-1-yl)benzene 14

Prepared according to general procedure A using 4-bromo-2,5-dimethoxybenzaldehyde and nitropropane with yield of 83% and taken forward without further characterization.

### (E)-1-iodo-2,5-dimethoxy-4-(2-nitrobut-1-en-1-yl)benzene 15

Prepared according to general procedure A using 4-iodo-2,5-dimethoxybenzaldehyde and nitropropane with yield of 66%.

¹H NMR (500 MHz, Chloroform-d) δ 8.10 (d, J=0.6 Hz, 1H), 7.35 (s, 1H), 6.74 (s, 1H), 3.84 (d, J=10.3 Hz, 6H), 2.80 (q, J=7.4 Hz, 2H), 1.27 (t, J=7.4 Hz, 3H).

### (E)-1-cyclopropyl-2,5-dimethoxy-4-(2-nitrobut-1-en-1-yl)benzene 16

Prepared according to general procedure A using 4-cyclopropyl-2,5-dimethoxybenzaldehyde and nitropropane with yield of 72%.

¹H NMR (400 MHz, Chloroform-d) δ 8.23 (s, 1H), 6.80 (s, 1H), 6.40 (s, 1H), 3.82 (d, J=17.0 Hz, 6H), 2.85 (q, J=7.4 Hz, 2H), 2.24 (tt, J=8.5, 5.3 Hz, 1H), 1.29 (t, J=7.4 Hz, 3H), 1.07-0.98 (m, 2H), 0.76-0.68 (m, 2H).

### (E)-1,4-dimethoxy-2-(2-nitrobut-1-en-1-yl)-5-(trifluoromethyl) benzene 17

Prepared according to general procedure A using 2,5-dimethoxy-4-(trifluoromethyl) benzaldehyde and nitropropane with yield of 89%.

¹H NMR (500 MHz, Chloroform-d) δ 8.09 (s, 1H), 7.13 (s, 1H), 6.91 (s, 1H), 3.87 (d, J=8.9 Hz, 6H), 2.78 (q, J=7.3 Hz, 2H), 1.26 (t, J=7.4 Hz, 3H).

### (E)-(2,5-dimethoxy-4-(2-nitrobut-1-en-1-yl)phenyl) (methyl)sulfane 18

Prepared according to general procedure A using 2,5-dimethoxy-4-(methylthio)benzaldehyde and nitropropane with yield of 79%.

¹H NMR (500 MHz, Chloroform-d) δ 8.26 (s, 1H), 6.81 (s, 1H), 6.74 (s, 1H), 3.89 (d, J=1.7 Hz, 6H), 2.87 (q, J=7.4 Hz, 2H), 2.51 (s, 3H), 1.31 (t, J=7.4 Hz, 3H).

### (E)-(2,5-dimethoxy-4-(2-nitrobut-1-en-1-yl)phenyl) (ethyl)sulfane 19

Prepared according to general procedure A using 4-(ethylthio)-2,5-dimethoxybenzaldehyde and nitropropane with yield of 69%.

¹H NMR (400 MHz, Chloroform-d) δ 8.24-8.20 (m, 1H), 6.81 (d, J=6.9 Hz, 2H), 3.86 (d, J=4.2 Hz, 6H), 2.98 (q, J=7.4 Hz, 2H), 2.85 (q, J=7.4 Hz, 2H), 1.38 (t, J=7.4 Hz, 3H), 1.29 (t, J=7.4 Hz, 3H).

### (E)-1-ethyl-2,5-dimethoxy-4-(2-nitrobut-1-en-1-yl)benzene 20

Prepared according to general procedure A using commercially available 4-ethyl-2,5-dimethoxybenzaldehyde and nitropropane with yield of 66%.

### (E)-1-ethoxy-4-methoxy-5-(2-nitrobut-1-en-1-yl)-2-(trifluoromethyl)benzene 21

Prepared from 5-ethoxy-2-methoxy-4-(trifluoromethyl)benzaldehyde 10 using general procedure A with a yield of 50%.

### (E)-1,4-dimethoxy-2-(methoxymethyl)-5-(2-nitrobut-1-en-1-yl)benzene 22

Prepared from 2,5-dimethoxy-4-(methoxymethyl)benzaldehyde 12 using general procedure A with a yield of 93%.

¹H NMR (400 MHz, CDCl₃) δ 8.22 (s, 2H), 7.04 (s, 2H), 6.79 (s, 2H), 4.52 (s, 4H), 3.86 (s, 3H), 3.81 (s, 3H), 3.48 (s, 3H), 2.83 (q, J=7.4 Hz, 2H), 1.27 (t, J=7.4 Hz, 3H).

### (E)-1-(2-cyclopropyl-2-nitrovinyl)-2,5-dimethoxy-4-methylbenzene 23

Prepared according to general procedure A from 2,5-dimethoxy-4-methylbenzaldehyde and (nitromethyl)cyclopropane with a yield of 80% and taken forward without further characterization.

### (E)-1-chloro-2,5-dimethoxy-4-(2-nitrobut-1-en-1-yl)benzene 24

Prepared according to general procedure A using 4-chloro-2,5-dimethoxybenzaldehyde and nitropropane with yield of 92% and taken forward without further characterization.

### (E)-1,4-dimethoxy-2-(2-nitrobut-1-en-1-yl)-5-propylbenzene 25

Prepared according to general procedure A using 2,5-dimethoxy-4-propylbenzaldehyde and nitropropane with yield of 73%.

¹H NMR (400 MHz, CDCl₃) δ 8.27 (s, 1H), 6.80 (d, J=15.4 Hz, 2H), 3.95-3.72 (m, 6H), 2.97-2.78 (m, 2H), 2.75-2.49 (m, 2H), 1.72-1.52 (m, 2H), 1.31 (t, J=7.3 Hz, 3H), 1.00 (t, J=7.4 Hz, 3H).

¹³C NMR (400 MHz, CDCl₃) δ 152.51, 152.13, 151.29, 135.64, 129.58, 118.85, 113.23, 111.36, 56.13, 55.91, 32.70, 22.99, 21.10, 14.05, 12.47.

### (E)-1,4-dimethoxy-2-(2-nitropent-1-en-1-yl)-5-propylbenzene 26

Prepared according to general procedure A using 2,5-dimethoxy-4-propylbenzaldehyde and nitrobutane with yield of 79%.

¹H NMR (400 MHz, CDCl₃) δ 8.29 (s, 1H), 6.79 (d, J=15.6 Hz, 2H), 3.95-3.74 (m, 6H), 2.99-2.75 (m, 2H), 2.73-2.54 (m, 2H), 1.79-1.50 (m, 4H), 1.13-0.92 (m, 6H). ¹³C NMR (400 MHz, CDCl₃) δ 152.59, 151.27, 151.02, 135.62, 129.74, 118.88, 113.24, 111.24, 56.15, 55.87, 32.71, 29.57, 22.98, 21.49, 14.06(2C).

### Example 18. Preparation of Ariadne (32) Analogs

### 1-(4-(ethylthio)-2,5-dimethoxyphenyl)butan-2-amine 27

Prepared from (E)-(2,5-dimethoxy-4-(2-nitrobut-1-en-1-yl)phenyl) (ethyl)sulfane using general procedure B with yield of 23%.

¹H NMR (400 MHz, Chloroform-d) δ 6.84 (s, 1H), 6.69 (s, 1H), 3.84 (s, 3H), 3.78 (s, 3H), 2.91 (q, J=7.4 Hz, 3H), 2.79 (dd, J=13.1, 4.6 Hz, 1H), 2.43 (dd, J=13.1, 8.6 Hz, 1H), 1.57-1.45 (m, 1H), 1.36 (dd, J=14.2, 6.9 Hz, 2H), 1.29 (t, J=7.4 Hz, 4H), 0.97 (t, J=7.4 Hz, 3H). ¹³C NMR (101 MHz, Chloroform-d) δ 151.86, 151.81, 127.46, 122.07, 114.17, 113.88, 56.46, 56.15, 52.92, 38.77, 30.57, 26.93, 14.29, 10.63. LRMS (APCI+) calcd. For C₁₄H₂₃NO₂S [M+H]⁺ 270.1, found 270.1.

### 1-(2,5-dimethoxy-4-(methylthio)phenyl)butan-2-amine 28

Prepared from (E)-(2,5-dimethoxy-4-(2-nitrobut-1-en-1-yl)phenyl) (methyl)sulfane using general procedure B with yield of 70%.

¹H NMR (400 MHz, Chloroform-d) δ 6.77 (s, 1H), 6.68 (s, 1H), 3.85 (s, 3H), 3.80 (s, 3H), 2.92 (m, J=8.5, 7.4, 4.9 Hz, 1H), 2.84-2.75 (m, 1H), 2.44 (s, 4H), 1.52 (m, 1H), 1.36 (m, 1H), 0.98 (t, J=7.4 Hz, 3H). LRMS (APCI+) calcd. For C₁₄H₂₃NO₂S [M+H]⁺ 256.1, found 256.1.

### 1-(2,5-dimethoxy-4-(trifluoromethyl) phenyl) butan-2-amine 29

Prepared from (E)-1,4-dimethoxy-2-(2-nitrobut-1-en-1-yl)-5-(trifluoromethyl)benzene using general procedure C with yield of 24%.

¹H NMR (500 MHz, DMSO-d6) δ 7.08 (d, J=2.0 Hz, 2H), 3.82 (s, 3H), 3.77 (s, 3H), 2.79 (tt, J=7.6, 5.0 Hz, 1H), 2.71 (dd, J=12.8, 5.4 Hz, 1H), 2.46 (dd, J=12.9, 7.8 Hz, 1H), 1.40-1.30 (m, 1H), 1.24 (br s, 2H), 1.22-1.16 (m, 1H), 0.88 (t, J=7.4 Hz, 3H). ¹³C NMR (126 MHz, DMSO) δ 150.71, 150.55(m), 134.72, 123.7(m), 116.17, 114.6(m), 108.8(m), 56.41, 56.05, 52.36, 38.46, 30.23, 10.40. LRMS (APCI+) calcd. For C₁₃H₁₈F₃NO₂ [M+H]⁺ 278.1, found 278.1.

### 1-(4-cyclopropyl-2,5-dimethoxyphenyl)butan-2-amine 30

Prepared from (E)-1-cyclopropyl-2,5-dimethoxy-4-(2-nitrobut-1-en-1-yl)benzene using general procedure C in yield of 44%.

¹H NMR (400 MHz, Methanol-d4) δ 6.71 (s, 1H), 6.43 (s, 1H), 3.80 (s, 3H), 3.73 (s, 3H), 2.89 (ddt, J=8.1, 7.1, 5.3 Hz, 1H), 2.77 (dd, J=13.1, 5.2 Hz, 1H), 2.45 (dd, J=13.1, 8.1 Hz, 1H), 2.12 (tt, J=8.6, 5.4 Hz, 1H), 1.56-1.30 (m, 2H), 0.97 (t, J=7.5 Hz, 3H), 0.91-0.85 (m, 2H), 0.67-0.58 (m, 2H). LRMS (APCI+) calcd. For C₁₅H₂₃NO₂ [M+H]⁺ 250.2, found 250.1.

### 1-(4-bromo-2,5-dimethoxyphenyl) butan-2-amine 31

Prepared from (E)-1-bromo-2,5-dimethoxy-4-(2-nitrobut-1-en-1-yl)benzene using general procedure C with yield of 90%.

¹H NMR (500 MHz, Chloroform-d) δ 7.03 (s, 1H), 6.75 (s, 1H), 3.84 (s, 3H), 3.76 (s, 3H), 2.92 (s, 1H), 2.78 (dd, J=13.1, 4.6 Hz, 1H), 2.42 (dd, J=13.1, 8.6 Hz, 1H), 1.55-1.45 (m, 1H), 1.34 (dt, J=13.5, 7.3 Hz, 1H), 0.97 (t, J=7.5 Hz, 3H). ¹³C NMR (126 MHz, Chloroform-d) δ 152.22, 149.78, 128.60, 115.89, 115.41, 108.90, 56.97, 56.09, 52.69, 38.84, 10.61. LRMS (APCI+) calcd. For C₁₂H₁₈BrNO₂ [M+H]⁺ 288.1, found 288.1.

### 1-(2,5-dimethoxy-4-methylphenyl)butan-2-amine 32

Prepared from (E)-1,4-dimethoxy-2-methyl-5-(2-nitrobut-1-en-1-yl)benzene using general procedure C with yield of 92%.

¹H NMR (400 MHz, Chloroform-d) δ 6.67 (d, J=11.3 Hz, 2H), 3.77 (d, J=8.0 Hz, 6H), 3.00-2.90 (m, 1H), 2.81 (dd, J=13.2, 4.6 Hz, 1H), 2.46 (dd, J=13.2, 8.6 Hz, 1H), 2.26 (s, 2H), 2.21 (s, 3H), 1.61-1.46 (m, 1H), 1.39 (dt, J=13.6, 7.3 Hz, 1H), 0.98 (t, J=7.4 Hz, 3H). LRMS (APCI+) calcd. For C₁₃H₂₁NO₂ [M+H]⁺ 224.2, found 224.1.

### 1-(2,5-dimethoxy-4-(phenylthio)phenyl)butan-2-amine 33

Prepared from (E)-(2,5-dimethoxy-4-(2-nitrobut-1-en-1-yl)phenyl) (phenyl)sulfane using general procedure B with yield of 19%.

¹H NMR (400 MHz, Methanol-d4) δ 7.34-7.18 (m, 5H), 6.86 (s, 1H), 6.74 (s, 1H), 3.77 (s, 3H), 3.63 (s, 3H), 2.97-2.92 (m, 1H), 2.83 (dd, J=13.2, 5.3 Hz, 1H), 2.53 (dd, J=13.1, 8.0 Hz, 1H), 1.53-1.47 (m, 1H), 1.43-1.36 (m, 1H), 0.98 (t, J=7.4 Hz, 3H). LRMS (APCI+) calcd. For C₁H₂₃NO₂S [M+H]⁺ 318.4, found 318.3.

### 1-(2,5-dimethoxy-4-(methylsulfonyl)phenyl)butan-2-amine trifluoroacetate 34

1-(2,5-dimethoxy-4-(methylthio)phenyl)butan-2-amine (25 mg, 98 umol, 1eq) was dissolved in 2 ml ethyl acetate. Vial was cooled in dry ice/acetonitrile bath and mCPBA was added in one portion (71 mg, 411umol, 4.2 eq). Reaction reached room temperature and stirred 20 hours. Reaction mixture was diluted with ethyl acetate, washed with sodium sulfite (sat., 2×5 mL), sodium bicarbonate (sat., 1×5 mL), and water (1×5 mL). The extract was dried over Na2SO4, filtered and evaporated to dryness. The resulting oily residue was purified by addition of trifluoroacetic acid and methanol and elutied through Thermo Scientific™ HyperSep™ C18 cartridge and evaporated to give product in 46% yield.

¹H NMR (400 MHz, Methanol-d4) δ 7.47 (s, 2H), 7.16 (s, 2H), 3.97 (s, 6H), 3.89 (s, 6H), 3.52-3.41 (m, 2H), 3.23 (s, 6H), 3.07 (d, J=6.0 Hz, 2H), 2.95 (dd, J=13.7, 7.6 Hz, 2H), 1.76-1.60 (m, 4H), 1.09-1.04 ¹³C NMR (101 MHz, Methanol-d4) δ 151.32, 132.32 (s), 127.52, 116.37, 110.53, 56.01, 55.19, 52.88, 41.82, 33.23, 25.45, 8.46. LRMS (APCI+) calcd. For C₁₃H₂₁NO₄S [M+H]⁺ 288.1, found 288.6.

### 1-(4-iodo-2,5-dimethoxyphenyl)butan-2-amine 35

Prepared from (E)-1-iodo-2,5-dimethoxy-4-(2-nitrobut-1-en-1-yl)benzene using general procedure C with a yield of 82%.

¹H NMR (400 MHz, CDCl₃) δ 6.68 (d, J=5.6 Hz, 4H), 3.78 (d, J=6.1 Hz, 11H), 3.05 (ddd, J=13.0, 7.6, 5.4 Hz, 2H), 2.84 (dd, J=13.3, 5.2 Hz, 2H), 2.57 (dd, J=13.3, 8.2 Hz, 3H), 1.61-1.43 (m, 4H), 1.00 (t, J=7.5 Hz, 6H). LRMS (APCI+) calcd. For C₁₂H₁₈INO₂ [M+H]⁺ 336.0, found 336.2

### 1-(4-ethyl-2,5-dimethoxyphenyl) butan-2-amine 36

Prepared from (E)-1-ethyl-2,5-dimethoxy-4-(2-nitrobut-1-en-1-yl)benzene using general procedure B. Freebase was dissolved in anhydrous diethyl ether and treated with 2N HCl in ether and filtered to produce hydrochloride salt in 55% total yield. 1H NMR (300 MHz, MeOD) δ 6.77 (d, J=15.1 Hz, 2H), 3.78 (d, J=6.9 Hz, 6H), 3.37 (dd, J=13.2, 6.5 Hz, 1H), 2.88 (ddd, J=21.2, 13.8, 6.7 Hz, 2H), 2.59 (q, J=7.5 Hz, 2H), 1.74-1.60 (m, 2H), 1.14 (t, J=7.5 Hz, 3H), 1.03 (t, J=7.5 Hz, 3H) LRMS (APCI+) calcd. For C₁₄H₂₃NO₂ [M+H]⁺ 238.2, found 238.7

### 1-(5-ethoxy-2-methoxy-4-(trifluoromethyl) phenyl) butan-2-amine 37

Prepared from (E)-1-ethoxy-4-methoxy-5-(2-nitrobut-1-en-1-yl)-2-(trifluoromethyl)benzene, following general procedure C in a yield of 56%

¹³C NMR (101 MHz, CDCl₃) δ 151.12 (s), 150.59 (s), 133.44 (s), 125.04 (s), 122.33 (s), 117.24 (s), 109.16 (dd), 65.44 (s), 55.97 (s), 52.72 (s), 38.69 (s), 30.37 (s), 14.76 (s), 10.50 (s). LRMS (APCI+) calcd. For C₁₄H₂₀F₃NO₂ [M+H]⁺ 292.1, found 292.4.

### 1-(2,5-dimethoxy-4-(methoxymethyl)phenyl)butan-2-amine 38

Prepared from (E)-1,4-dimethoxy-2-(methoxymethyl)-5-(2-nitrobut-1-en-1-yl)benzene according to General procedure B with yield of 20%.

¹H NMR (400 MHz, CDCl₃) δ 6.90 (s, 1H), 6.70 (s, 1H), 4.47 (s, 2H), 3.79 (s, 6H), 3.43 (s, 3H), 3.01-2.88 (m, 1H), 2.82 (dd, 1H), 2.46 (dd, 1H), 1.67 (bs, 2H), 1.57-1.44 (m, 1H), 1.42-1.26 (m, 1H), 0.97 (t, J=7.4 Hz, 3H). ¹³C NMR (101 MHz, CDCl₃) δ 151.93, 150.95, 128.27, 125.25, 114.20, 112.03, 69.52, 58.58, 56.34, 56.13, 53.08, 38.93, 30.60, 10.74. LRMS (APCI+) calcd. For C₁₄H₂₃NO₃ [M+H]⁺ 254.2, found 254.4

### 1-cyclopropyl-2-(2,5-dimethoxy-4-methylphenyl) ethan-1-amine 39

Prepared from ((E)-1-(2-cyclopropyl-2-nitrovinyl)-2,5-dimethoxy-4-methylbenzene using general procedure B with yield of 41% (as HCl salt).

¹H NMR (400 MHz, MeOD) 6.82 (s, 1H), 6.79 (s, 1H), 3.90-3.73 (m, 6H), 3.39-3.28 (m, 4H), 3.12-2.93 (m, 2H), 2.72-2.60 (m, 1H), 2.21 (s, 3H), 1.08-0.91 (m, 1H), 0.72-0.54 (m, 1H), 0.47-0.36 (m, 1H), 0.23-0.13 (m, 1H). ¹³C NMR (400 MHz, MeOD) δ 151.71, 151.31, 126.40, 121.40, 113.69, 113.46, 57.87, 55.12, 54.88, 34.25, 14.89, 13.37, 3.38, 2.75. LRMS (APCI+) calcd. For C₁₄H₂₁NO₂ [M+H]⁺ 236.2, found 236.2.

### 1-(2,5-dimethoxy-4-methylphenyl)pentan-2-amine 40

Prepared from 2,5-dimethoxy-4-methylbenzaldehyde using general procedures A directly followed by B with yield of 2% (over 2 steps).

¹H NMR (500 MHz, MeOD) δ 6.77 (s, 1H), 6.70 (s, 1H), 3.83-3.72 (m, 6H), 3.04-2.97 (m, 1H), 2.81 (dd, J=13.1, 5.2 Hz, 1H), 2.46 (dd, J=13.1, 8.2 Hz, 1H), 1.54-1.28 (m, 4H), 1.00-0.91 (m, 3H). 13C NMR (500 MHz, MeOD) δ 151.48, 151.42, 125.03, 124.97, 113.50, 113.46, 61.42, 55.04, 50.78, 38.79, 37.76, 28.78, 14.84, 13.11.

### 1-(2,6-dimethoxy-4-methylphenyl)butan-2-amine 41

Prepared from 2,6-dimethoxy-4-methylbenzaldehyde using general procedures A directly followed by B with yield of 1.3% (over 2 steps).

¹H NMR (500 MHz, MeOD) δ 6.47 (s, 2H), 3.80 (s, 6H), 2.98-2.90 (m, 1H), 2.76 (dd, J=13.0, 5.2 Hz, 1H), 2.61 (dd, J=13.0, 8.1 Hz, 1H), 2.34 (s, 3H), 1.50-1.34 (m, 4H), 0.97-0.90 (m, 3H). ¹³C NMR (500 MHz, MeOD) δ 158.36(2C), 137.52, 112.07, 104.22(2C), 54.56(2C), 50.90, 38.81, 29.78, 20.66, 18.96, 13.09.

### 1-(4-chloro-2,5-dimethoxyphenyl)butan-2-amine 42

Prepared from (E)-1-chloro-2,5-dimethoxy-4-(2-nitrobut-1-en-1-yl)benzene using general procedures A with yield of 51%.

¹H NMR (400 MHz, MeOD) δ 7.06 (s, 1H), 7.00 (s, 1H), 3.87 (s, 3H), 3.84 (s, 3H), 3.46-3.37 (m, 1H), 3.01 (dd, J=13.9, 6.2 Hz, 1H), 2.89 (dd, J=13.8, 7.4 Hz, 1H), 1.76-1.61 (m, 2H), 1.07 (t, J=7.5 Hz, 3H). ¹³C NMR (400 MHz, MeOD) δ 151.86, 149.26, 123.46, 121.57, 115.91, 113.05, 56.03, 55.23, 53.25, 32.89, 25.29, 8.57.

### 1-(2,5-dimethoxy-4-propylphenyl)butan-2-amine 43

Prepared from (E)-1,4-dimethoxy-2-(2-nitroprop-1-en-1-yl)-5-propylbenzene using general procedures A with yield of 54% (as the HCl salt). ¹H NMR (400 MHz, MeOD) δ 6.82 (s, 1H), 6.78 (s, 1H), 3.82 (s, 3H), 3.80 (s, 3H), 3.57 (h, J=6.7 Hz, 1H), 2.95 (dd, J=13.5, 6.7 Hz, 1H), 2.85 (dd, J=13.5, 7.0 Hz, 1H), 2.63-2.56 (m, 2H), 1.68-1.54 (m, 2H), 1.29 (d, J=6.6 Hz, 3H), 0.96 (t, J=7.4 Hz, 3H). ¹³C NMR (400 MHz, MeOD) δ 151.54, 151.36, 131.13, 121.57, 113.84, 112.96, 55.17, 54.92, 48.08, 35.32, 32.04, 22.98, 17.23, 12.92.

### 1-(2,5-dimethoxy-4-propylphenyl)pentan-2-amine 44

Prepared from (E)-1,4-dimethoxy-2-(2-nitroprop-1-en-1-yl)-5-propylbenzene using general procedures A with yield of 41% (as the HCl salt). ¹H NMR (400 MHz, MeOD) δ 6.82 (s, 1H), 6.79 (s, 1H), 3.83 (s, 3H), 3.80 (s, 3H), 3.51-3.42 (m, 1H), 2.99 (dd, J=13.9, 6.0 Hz, 1H), 2.85 (dd, J=13.8, 7.4 Hz, 1H), 2.63-2.55 (m, 2H), 1.68-1.56 (m, 4H), 1.54-1.40 (m, 2H), 1.03-0.92 (m, 6H). ¹³C NMR (400 MHz, MeOD) δ 151.57, 151.41, 131.16, 121.49, 113.88, 112.99, 55.18, 54.93, 51.94, 34.47, 33.49, 32.04, 22.98, 18.19, 12.92, 12.65.

These methods can be used to prepare the rest of the compounds described herein through suitable modification of reagents and starting materials.

### Example 19. Pharmacological characterization of Ariadne 32 analogs

Selectivity of Ariadne for CNS targets

In SafetyScreen44 (Panlabs) panel of 44 CNS targets, Ariadne (32, at a concentration of 10 μM) demonstrated ≥50% replacement of standard ligand at only two targets, 5-HT2A and 5-HT2B. Ariadne (32) registered 72% inhibition at only 5-HT2A and 5-HT2B receptors.

TABLE 1

| Assay Name | Species | Conc. | % Inh. |
|---|---|---|---|
| Serotonin (5-Hydroxytryptamine) | hum | 10 μM | 72 |
| 5-HT_{2A} | | | |
| Serotonin (5-Hydroxytryptamine) | hum | 10 μM | 72 |
| 5-HT_{2B} | | | |

Other targets included with less than ≥50% stimulation at a concentration of 10 μM: Acetylcholinesterase, COX-1, COX-2, MAO-A, PDE3A, PDE4D2, Protein Tyrosine Kinase (LCK), Adenosine A2A, Adrenergic α1A, Adrenergic α2A, Adrenergic β1, Adrenergic β2, Androgen (Testosterone), Calcium Channel L-Type (Dihydropyridine), CB1, CB2, Cholecystokinin CCK1 (CCKA), Dopamine D1, Dopamine D2S, Endothelin ETA, GABAA (Flunitrazepam, Central), Glucocorticoid, Glutamate-NMDA (Agonism), Histamine H1, Histamine H2, Muscarinic M1, Muscarinic M2, Muscarinic M3, Nicotinic Acetylcholine α4β2, (Cytisine), DOP, KOP, MOP, Potassium Channel [KA], Potassium Channel hERG, Serotonin (5-Hydroxytryptamine) 5-HT1A, 5-HT1B, 5-HT3, Na-channel site 2, DAT, NET, SERT, Vasopressin V1A

TABLE 2
Modification in the 4-position improves 5-HT2A/5-HT2B selectivity
profile. EC₅₀ values shown were obtained in an IP1 agonist functional
assay. Selectivity for 5HT2A versus 5HT2B receptor is based on relative
agonism potencies expressed as EC₅₀ values.

| | | | Selectivity |
|---|---|---|---|
| | EC₅₀ | EC₅₀ | (EC₅₀ 2B ÷ |
| | 5-HT2A | 5-HT2B | EC₅₀ 2A) |
| Ariadne (32) | 1.3E−06M | >3.2E−05M | 25x |
| (29) | 3.4E−08M | 5.9E−06M | 174x |
| (38) | 3.8E−6M | N.C. | N.C. |

### Example 20. Behavioral Assays in Mice

Forced Swim Test Protocol

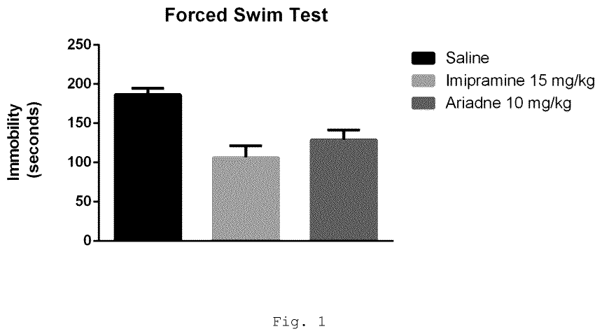

Mice (C57bl/6J, 8-15 weeks, 29-35 g) were weighed and moved to the testing room 30 minutes prior to experimentation. Mice were administered 0.85% saline (s.c., 5 mice), imipramine 15 mg/kg (i.p., 10 mice), or Ariadne (32) 10 mg/kg (s.c., 9 mice). 15 minutes post injection the mice were moved to a forced swim cylinder with water kept at 23.8-25.0 degrees celsius. Mice were allowed to swim for 6 minutes after which they were removed, dried, and returned to their home cage. The last four minutes of the forced swim video footage was analyzed with Noldus FST scoring software. The mouse's view of the FST cylinder was obstructed prior to starting the forced swim test. The results are shown in FIG. 1.

Head Twitch Response Protocol

Mice (C57bl/6J, 8-15 weeks, 26-33 g) were weighed and moved to the testing room 30 minutes prior to experimentation. Mice were administered the experimental compound at a selected dose s.c. and then immediately moved to a new cage. Before beginning the test, mice were allowed to remain in this cage for a set amount of time ranging 0-10 min based on the compound. The head twitch assay was performed by a trained observer watching the mouse for a 10 min duration and recording the total number of head twitches observed.

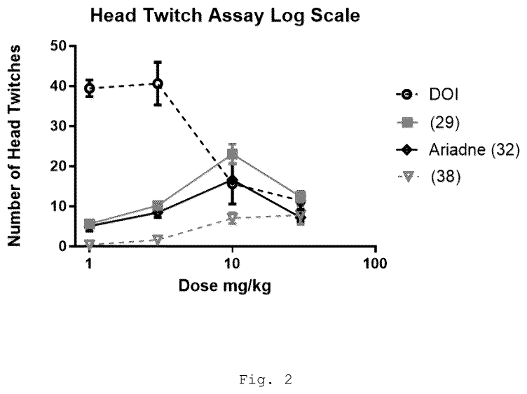

DOI was tested at the doses of 1, 3, 10 and 30 mg/kg (s.c., 5 mice per dose) and testing began immediately after administration of the compound. Ariadne (32), (29) and (38) were tested at the doses of 1, 3, 10, and 30 mg/kg (s.c., 5 mice per dose) and there was a 10 min interval between treatment and testing. The results are shown in FIG. 2.

### DISCUSSION

In this invention we demonstrate that Ariadne and its novel analogs act as 5HT2A receptor agonists and are highly selective across a broad panel of molecular targets. We also show that the novel compounds claimed here provide increased separation between the 5HT2A and 5HT2B receptor activities in terms of both potency and efficacy of downstream signaling. Activation of the latter target is associated with adverse cardiac effects and thus these new molecular entities provide increased safety and therapeutic index. The claimed compounds show antidepressant-like activity while exhibiting limited hallucinogenic-like activity in mice. These preclinical results correlate with the observations with Ariadne in humans, thus providing a preclinical model for assessing hallucinogenic efficacy of novel compounds. The disclosed compounds provide important candidates for the development of new therapeutics for a number of disorders claimed herein.

### REFERENCES

Alles, G. A. Some relations between chemical structure and physiological action of mescaline and related compounds. In: Abramson H A, ed. Neuropharmacology. New York, NY: Josiah Macy Jr. Foundation; (1959).
Barfknecht, C. F. & Nichols, D. E. J Med Chem. 14, 370-372 (1971).
Bogenschutz, M. P. et al. J Psychopharmacol. 29, 289-299 (2015).
Cameron, L. P. et al. Nature. 589, 474-479 (2021).
Davis, A. K. et al. JAMA Psychiatry. e203285 (2020).
Feng, Z. et al. Bioorg Med Chem Lett. 17, 2998-3002 (2007).
Foster, T. P. & Nichols, C. D. International Patent Application WO 2018/204359 A1, Published: Nov. 8, 2018.
Garcia-Romeu, A. et al. Curr Drug Abuse Rev. 7, 157-164 (2014).
Griffiths, R. R. et al. J Psychopharmacol. 30, 1181-1197 (2016).
Hofmann, A. Chapter 3 Chemical modifications of LSD, in LSD My Problem Child, Ed., MAPS (2009).
Kuypers, K. P. et al. J Psychopharmacol. 33, 1039-1057 (2019).
May, J. et al. Australian Patent Application AU 199961326 B2, Published: Apr. 10, 2000.
Nichols, D. E. Psychedelics. Pharmacol Rev. 68, 264-355 (2016).
Partyka, R. A. et al. U.S. Pat. No. 4,105,695, Published: Aug. 8, 1978.
Sewell, R. A. et al. Neurology. 66, 1920-1922 (2006).
Shulgin, A. T. J Med Chem. 11, 186-187 (1968).
Standridge, R. T. et al. J Med Chem. 19, 1400-1404 (1976).
Terry, M. et al. Journal of Archaeological Science. 33, 1017-1021 (2006).

1. A compound having the structure: 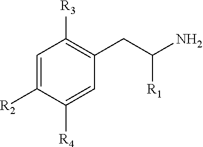
wherein
R₁ is —(C₂-C₁₂ alkyl), -(alkenyl), -(alkynyl), -(cycloalkyl);
R₂ is H, halogen, —NO₂, —CN, —CF₃, —CF₂H, —CH₂OCH₃, —CF₂CH₃, —CF₂OCH₃, -(alkyl), -(alkenyl), -(alkynyl), -(cycloalkyl), -(cycloalkylalkyl), -(heteroalkyl), -(heterocycle), -(heterocycloalkyl), -(aryl), -(heteroaryl), -(hydroxyalkyl), -(haloalkyl), -(alkylaryl), —OH, —O-(alkyl), —O-(alkenyl), —O-(alkynyl), —O-(haloalkyl), —O-(aryl), —O-(heteroaryl), —OCF₃, SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —N-(alkyl)₂, NH-(aryl), —NH-(heteroaryl), —CO₂H, —CO₂-(alkyl), —C(O)—NH₂, —C(O)—NH-(alkyl), —C(O)—NH-(aryl), —SO₂CH₃ or —Si(CH₃) ₃;
R₃ is —OCH₃, —OCH₂CH₃, —F or —Cl; and
R₄ is —OCH₃, —OCH₂CH₃ or —SCH₃;
wherein when R₁ is —CH₂CH₃, R₃ is —OCH₃, and R₄ is —OCH₃, then R₂ is other than H, —CH₃, —CH₂CH₃, —CH₂CH₂CH₃, —CH(CH₃)₂, —CH₂CH₂CH₂CH₃, —CH₂OH, —CH(OH) CH₃, —OH, —OCH₂CH₃, —OCH₂CH₂CH₃, —OCH(CH₃)₂, —SCH₃, —SCH₂CH₃, —SCH₂CH₂CH₃, —NO₂, —NH₂, —F, —Cl, —Br or —I,
or a pharmaceutically acceptable salt thereof.

2. A compound having the structure: 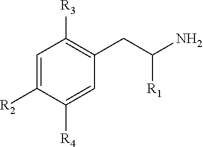
wherein
R₁ is —(C₃-C₁₂ alkyl), -(alkenyl), -(alkynyl), -(cycloalkyl);
R₂ is H, halogen, —NO₂, —CN, —CF₃, —CF₂H, —CH₂OCH₃, —CF₂CH₃, —CF₂OCH₃, -(alkyl), -(alkenyl), -(alkynyl), -(cycloalkyl), -(cycloalkylalkyl), -(heteroalkyl), -(heterocycle), -(heterocycloalkyl), -(aryl), -(heteroaryl), -(hydroxyalkyl), -(haloalkyl), -(alkylaryl), —OH, —O-(alkyl), —O-(alkenyl), —O-(alkynyl), —O-(haloalkyl), —O-(aryl), —O-(heteroaryl), —OCF₃, SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —N-(alkyl)₂, NH-(aryl), —NH-(heteroaryl), —CO₂H, —CO₂-(alkyl), —C(O)—NH₂, —C(O)—NH-(alkyl), —C(O)—NH-(aryl), —SO₂CH₃ or —Si(CH₃) ₃;
R₃ is —OCH₃, —OCH₂CH₃, —F or —Cl; and
R₄ is —OCH₃, —OCH₂CH₃ or —SCH₃,
or a pharmaceutically acceptable salt thereof.

3. The compound of claim 1, wherein
R₁ is —(C₂-C₁₂ alkyl), -(alkenyl), -(alkynyl), -(cycloalkyl);
R₂ is H, halogen, —NO₂, —CN, —CF₃, —CF₂H, -(alkyl), -(alkenyl), -(alkynyl), -(cycloalkyl), -(cycloalkylalkyl), -(heteroalkyl), -(heterocycle), -(heterocycloalkyl), -(aryl), -(heteroaryl), -(hydroxyalkyl), -(haloalkyl), -(alkylaryl), —OH, —O-(alkyl), —O-(alkenyl), —O-(alkynyl), —O-(haloalkyl), —O-(aryl), —O-(heteroaryl), —OCF₃, SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —N-(alkyl)₂, NH-(aryl), —NH-(heteroaryl), —CO₂H, —CO₂-(alkyl), —C(O)—NH₂, —C(O)—NH-(alkyl), —C(O)—NH-(aryl) or —Si(CH₃)₃;
R₃ is —OCH₃, —F or —Cl; and
R₄ is —OCH₃ or —SCH₃;
wherein when R₁ is —CH₂CH₃, R₃ is —OCH₃, and R₄ is —OCH₃, then R₂ is other than H, —CH₃, —CH₂CH₃, —CH₂CH₂CH₃, —CH(CH₃)₂, —CH₂CH₂CH₂CH₃, —CH₂OH, —CH(OH) CH₃, —OH, —OCH₂CH₃, —OCH₂CH₂CH₃, —OCH(CH₃)₂, —SCH₃, —SCH₂CH₃, —SCH₂CH₂CH₃, —NO₂, —NH₂, —F, —Cl, —Br or —I,
or a pharmaceutically acceptable salt thereof.

4. The compound of claim 2, wherein
R₁ is —(C₃-C₁₂ alkyl), -(alkenyl), -(alkynyl), -(cycloalkyl);
R₂ is H, halogen, —NO₂, —CN, —CF₃, —CF₂H, -(alkyl), -(alkenyl), -(alkynyl), -(cycloalkyl), -(cycloalkylalkyl), -(heteroalkyl), -(heterocycle), -(heterocycloalkyl), -(aryl), -(heteroaryl), -(hydroxyalkyl), -(haloalkyl), -(alkylaryl), —OH, —O-(alkyl), —O-(alkenyl), —O-(alkynyl), —O-(haloalkyl), —O-(aryl), —O-(heteroaryl), —OCF₃, SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —N-(alkyl)₂, NH-(aryl), —NH-(heteroaryl), —CO₂H, —CO₂-(alkyl), —C(O)—NH₂, —C(O)—NH-(alkyl), —C(O)—NH-(aryl) or —Si(CH₃)₃;
R₃ is —OCH₃, —F or —Cl; and
R₄ is —OCH₃ or —SCH₃,
or a pharmaceutically acceptable salt thereof.

5. The compound of claim 1, wherein
R₁ is —(C₂-C₁₂ alkyl), -(alkenyl), -(alkynyl), -(cycloalkyl);
R₂ is H, —CN, —CF₃, —CH₂OCH₃, —CF₂CH₃, —CF₂OCH₃, -(alkyl), -(alkenyl), -(alkynyl), -(cycloalkyl), -(heterocycle), —S-(aryl), —SO₂CH₃ or —Si(CH₃)₃;
R₃ is —OCH₃, —OCH₂CH₃, —F or —Cl; and
R₄ is —OCH₃, —OCH₂CH₃ or —SCH₃.
wherein
R₁ is -(C₂-C₁₂ alkyl), -(alkenyl), -(alkynyl), -(cycloalkyl);
R₂ is H, —CN, —CF₃, -(alkyl), -(alkenyl), -(alkynyl), -(cycloalkyl), -(heterocycle), —S-(aryl) or —Si(CH₃)₃;
R₃ is —OCH₃, —F or —Cl; and
R₄ is —OCH₃ or —SCH₃, or
wherein
R₁ is —(C₃-C₁₂ alkyl), -(alkenyl), -(alkynyl), -(cycloalkyl);
R₂ is H, —CN, —CF₃, —CH₂OCH₃, —CF₂CH₃, —CF₂OCH₃, -(alkyl), -(alkenyl), -(alkynyl), -(cycloalkyl), -(heterocycle), —S-(aryl), —SO₂CH₃ or —Si(CH₃)₃;
R₃ is —OCH₃, —OCH₂CH₃, —F or —Cl; and
R₄ is —OCH₃, —OCH₂CH₃ or —SCH₃; or
R₁ is —(C₃-C₁₂ alkyl), -(alkenyl), -(alkynyl), -(cycloalkyl);
R₂ is H, —CN, —CF₃, -(alkyl), -(alkenyl), -(alkynyl), -(cycloalkyl), -(heterocycle), —S-(aryl) or —Si(CH₃)₃;
R₃ is —OCH₃, —F or —Cl; and
R₄ is —OCH₃ or —SCH₃, or
R₁ is —CH₂CH₃, —CH₂CH₂CH₃, —CH₂CH═CH₂ or cyclopropyl.

6-9. (canceled)

10. The compound of claim 9, wherein
R₁ is —CH₂CH₂CH₃, —CH₂CH═CH₂ or cyclopropyl; or
wherein
R₂ is —CN, —CF₃, —CF₂H, —CH₂OCH₃, —CF₂CH₃, —CF₂OCH₃, —CH₃, —CCH, -cyclopropyl, -cyclobutyl, 2-oxetanyl, —S- (phenyl) , —SO₂CH₃ or —Si (CH₃)₃; or
wherein
R₂is —CN, —CF₃, —CH₃, —CCH, -cyclopropyl, -cyclobutyl, 2-oxetanyl, —S-(phenyl) or —Si(CH₃)₃; or
wherein R₃ is —OCH₃, —F or —Cl; or
wherein R₃ is —OCH₂CH₃; or
wherein R₄ is —OCH₃ or —SCH₃; or
wherein R₄ is —OCH₂CH₃.

11-16 (canceled)

17. The compound of claim 1, wherein
R₁ is —CH₂CH₃, —CH₂CH₂CH₃, —CH₂CH═CH₂ or cyclopropyl;
R₂ is —CN, —CF₃, —CH₂OCH₃, —CF₂CH₃, —CF₂OCH₃, —CH₃, —CCH, -cyclopropyl, -cyclobutyl, 2-oxetanyl, —S-(phenyl), —SO₂CH₃ or —Si(CH₃) ₃;
R₃ is —OCH₃, —OCH₂CH₃, F or Cl, and
R₄ is —OCH₃, —OCH₂CH₃ or —SCH₃; or
wherein
R₁ is —CH₂CH₃, —CH₂CH₂CH₃, —CH₂CH═CH₂ or cyclopropyl;
R₂ is —CN, —CF₃, —CH₃, —CCH, -cyclopropyl, -cyclobutyl, 2-oxetanyl, —S-(phenyl) or —Si(CH₃)₃;
R₃ is —OCH₃, F or Cl, and
R₄ is —OCH₃ or —SCH₃;or
wherein
R₁ is —CH₂CH₂CH₃, —CH₂CH═CH₂ or cyclopropyl;
R₂ is —CN, —CF₃, —CH₂OCH₃, —CF₂CH₃, —CF₂OCH₃, —CH₃, —CCH, -cyclopropyl, -cyclobutyl, 2-oxetanyl, —S-(phenyl), —SO₂CH₃ or —Si(CH₃) ₃;
R₃ is —OCH₃, —OCH₂CH₃, F or Cl, and
R₄ is —OCH₃, —OCH₂CH₃, or —SCH₃; or
wherein
R₁ is —CH₂CH₂CH₃, —CH₂CH═CH₂ or cyclopropyl;
R₂ is —CN, —CF₃, —CH₃, —CCH, -cyclopropyl, -cyclobutyl, 2-oxetanyl, —S-(phenyl) or —Si(CH₃)₃;
R₃ is —OCH₃, F or Cl, and
R₄ is —OCH₃ or —SCH₃.

18-20. (canceled)

21. The compound of claim 1, having the structure: 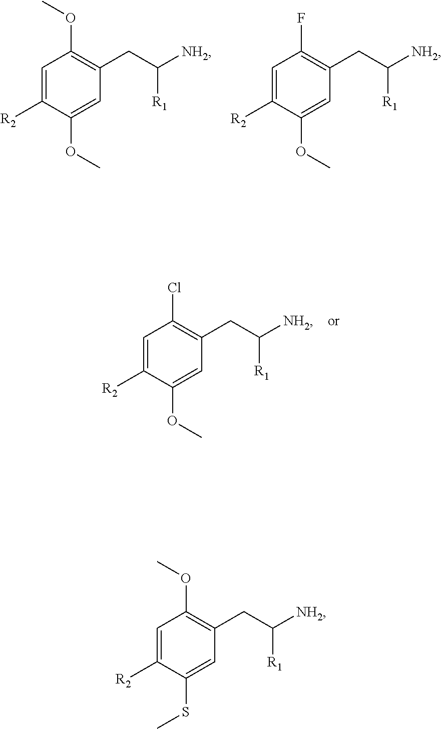
or a pharmaceutically acceptable salt thereof.

22-24. (canceled)

25. The compound of claim 1, having the structure: 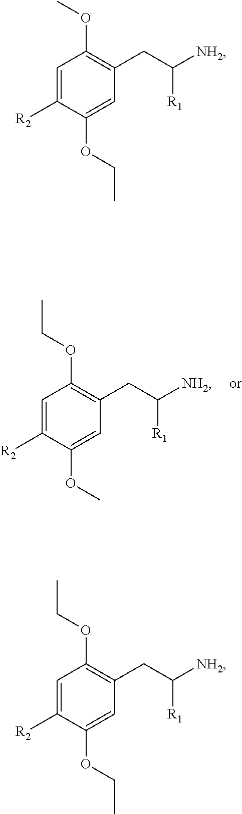
or a pharmaceutically acceptable salt thereof.

26-27 (canceled)

28. The compound of claim 25, wherein
R₁ is —CH₂CH₃, —CH₂CH₂CH₃, —CH₂CH═CH₂ or cyclopropyl, or
wherein
R₂ is H, —CN, —CF₃, -(alkyl), -(alkenyl), -(alkynyl), -(cycloalkyl), -(heterocycle), —S-(aryl) or —Si(CH₃)₃, or
wherein
R₂ is —CN, —CF₃, —CH₃, —CCH, -cyclopropyl, -cyclobutyl, 2-oxetanyl, —S-(phenyl) or —Si(CH₃)₃, or
R₂ is —CF₃, —CH₂OCH₃ or —CF₂OCH₃, or
wherein
R₂ is —CN or —CF₃, or
R₁ is —CH₂CH₃, —CH₂CH₂CH₃, —CH₂CH═CH₂ or cyclopropyl; and
R₂ is —CN or —CF₃, or R₁ is —CH₂CH₃, and R₂ is —CN or —CF₃, or
R₁ is —CH₂CH₂CH₃, and R₂ is —CN or —CF₃, or
R₁ is —CH₂CH═CH₂, and R₂ is —CN or —CF₃, or R₁ is cyclopropyl, and R₂ is —CN or —CF₃, or
wherein
R₂ is —CF₃, —CH₂OCH₃, —CF₂CH₃, —CF₂OCH₃ or —SO₂CH₃, or
R₁ is —CH₂CH₃, —CH₂CH₂CH₃, —CH₂CH═CH₂ or cyclopropyl; and
R₂ is —CF₃, —CH₂OCH₃, —CF₂CH₃, —CF₂OCH₃ or —SO₂CH₃, or
R₁ is —CH₂CH₃, and R₂ is CF₃, —CH₂OCH₃, —CF₂CH₃, —CF₂OCH₃ or —SO₂CH₃, or
R₁ is —CH₂CH₂CH₃, and R₂ is CF₃, —CH₂OCH₃, —CF₂CH₃, —CF₂OCH₃ or —SO₂CH₃, or
R₁ is —CH₂CH═CH₂, and R₂ is CF₃, —CH₂OCH₃, —CF₂CH₃, —CF₂OCH₃ or —SO₂CH₃, or
R₁ is cyclopropyl, and R₂ is CF₃, —CH₂OCH₃, —CF₂CH₃, —CF₂OCH₃ or —SO₂CH₃, or
wherein
R₁ is —CH₂CH₃; and
R₂ is —CF₃, —CH₂OCH₃ or —CF₂OCH₃, or
wherein
R₁ is —CH₂CH₃,
R₂ is —CF₃, —CH₂OCH₃ or —CF₂OCH₃,
R₃ is —OCH₃; and
R₄ is —OCH₃.

29-34. (canceled)

35. The compound of claim 1 having the structure: 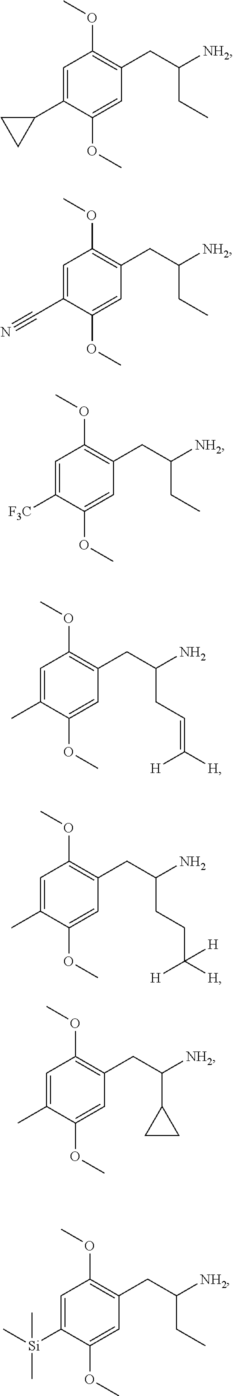 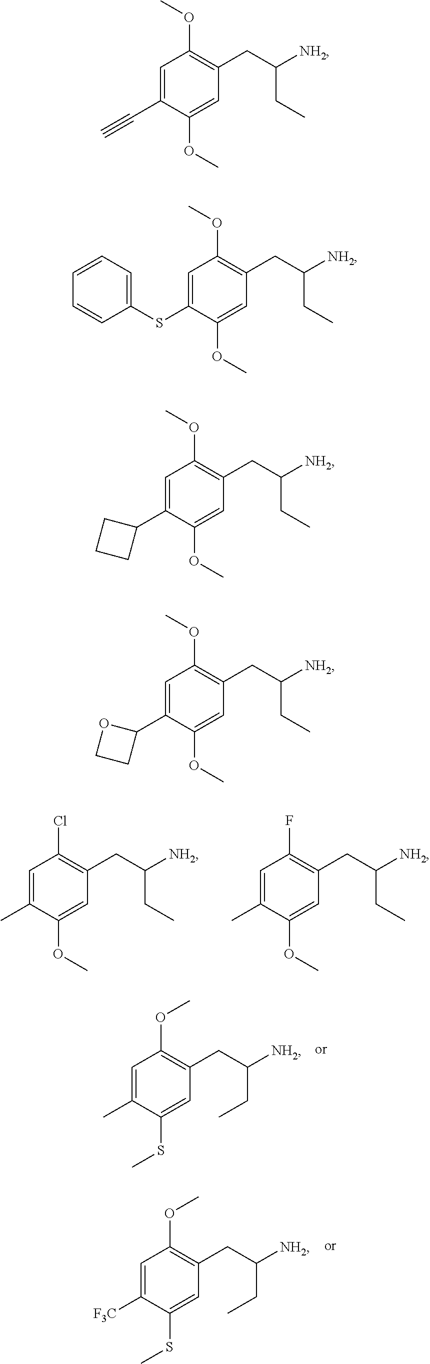 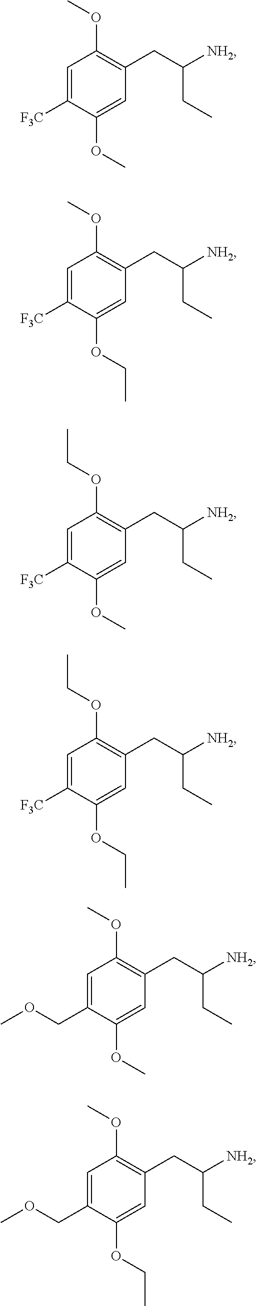 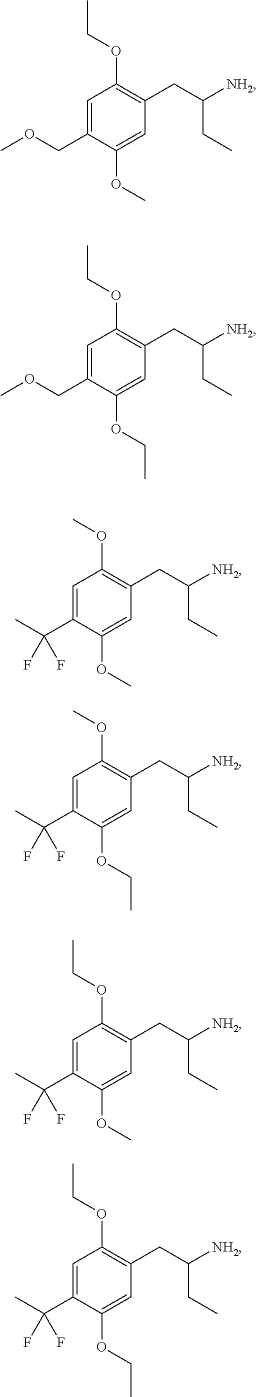 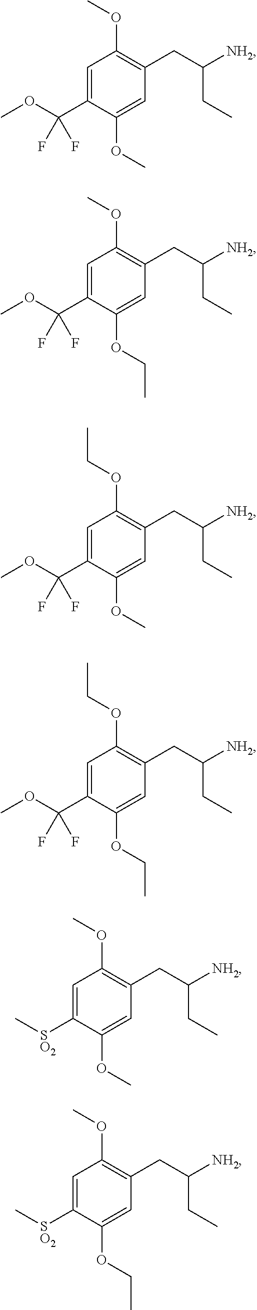 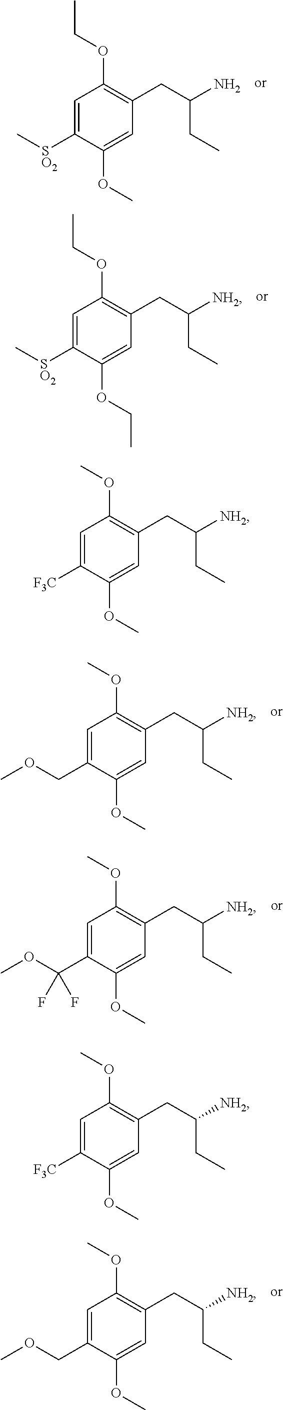 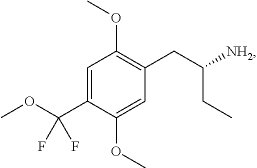 [SMILES: CC[C@@H](N)Cc1cc(OC)c(C(F)(F)OC)cc1OC]
or a pharmaceutically acceptable salt thereof.

36-39. (canceled)

40. A pharmaceutical composition comprising the compound of of claim 1 and a pharmaceutically acceptable carrier.

41. A method of activating 5HT2A receptor comprising contacting the 5HT2A receptor with the composition of claim 40, or
of selectively activating 5HT2A receptor compared to 5HT2B, comprising selectively contacting the 5HT2A receptor compared to 5HT2B with the composition of claim 40.

43. A method of treating a subject afflicted with Parkinson's disease comprising administering to the subject the composition of claim 40 comprising an effective amount of the compound, so as to thereby treat the subject afflicted with Parkinson's disease, or
of treating a subject afflicted with dementia or Alzheimer's disease comprising administering to the subject the compound of claim 40 comprising an effective amount of the compound, so as to thereby treat the subject afflicted with dementia or Alzheimer's disease, or
of treating a subject afflicted with attention deficit hyperactivity disorder (ADHD) comprising administering to the subject the compound of claim 40 comprising an effective amount of the compound, so as to thereby treat the subject afflicted with ADHD, or
of treating a subject afflicted with schizophrenia comprising administering to the subject the compound of claim 40 comprising an effective amount of the compound, so as to thereby treat the subject afflicted with schizophrenia, or
of treating a subject afflicted with depression, bipolar disorder, anxiety disorder, obsessive-compulsive disorder (OCD) or stress disorder comprising administering to the subject the composition of claim 40 comprising an effective amount of the compound, so as to thereby treat the subject afflicted with depression, bipolar disorder, anxiety disorder, obsessive-compulsive disorder (OCD) or stress disorder, or
of treating a subject afflicted with a substance use disorder comprising administering to the subject the compound of claim 40 comprising an effective amount of the compound, so as to thereby treat the subject afflicted with the substance use disorder
wherein the substance use disorder is opioid use disorder, alcohol use disorder or stimulant use disorder including nicotine use disorder,
wherein the substance is an opioid,
wherein the opioid is morphine, hydromorphone, oxymorphone, codeine, dihydrocodeine, hydrocodone, oxycodone, nalbuphine, butorphanol, etorphine, dihydroetorphine, levorphanol, metazocine, pentazocine, meptazinol, meperidine (pethidine), buprenorphine, methadone, tramadol, tapentadol, mitragynine, 3-deutero-mitragynine, 7-hydroxymitragynine, 3-deutero-7-hydroxymitragynine, mitragynine pseudoindoxyl or tianeptine, or wherein the opioid is fentanyl, sufentanil, alfentanil, furanylfentanyl, 3-methylfentanyl, valerylfentanyl, butyrylfentanyl, β-Hydroxythiofentanyl, acrylfentanyl or carfentanil, or
wherein the risk of relapse to the use of opioids, alcohol or stimulants is reduced, or wherein self-administration of an opioid, alcohol or stimulant is reduced, or
of treating a subject afflicted with opioid withdrawal symptoms comprising administering to the subject the composition of claim 40 comprising an effective amount of the compound, so as to thereby treat the subject afflicted with the opioid withdrawal symptoms,
wherein a symptom of substance use disorder is opioid withdrawal or mitigation of relapse to opioid use or SUD, or
of treating a subject afflicted with cluster headache, comprising administering to the subject the compound of claim 40 comprising an effective amount of the compound, so as to thereby treat the subject afflicted with cluster headache, or
of treating a subject afflicted with diabetic retinopathy, dry eyes, macular degeneration or glaucoma comprising administering to the subject the compound of claim 40 comprising an effective amount of the compound, so as to thereby treat the subject afflicted with diabetic retinopathy, dry eyes, macular degeneration, or glaucoma, or
of treating a subject afflicted with catatonia, comprising administering to the subject the compound of claim 40 comprising an effective amount of the compound, so as to thereby treat the subject afflicted with catatonia, or
of enhancing alertness in a subject, comprising administering to the subject the compound of claim 40 comprising an effective amount of the compound, so as to thereby enhance alertness in a subject,
wherein the effective amount of the compound administered to the subject does not induce a stimulant effect, or wherein the effective amount of the compound administered to the subject does not induce a hallucinogenic effect, or wherein the effective amount of the compound administered to the subject does not induce a stimulant effect and a hallucinogenic effect,
preferably wherein the subject is a mammal, further prefeably wherein the mammal is a human,
wherein the effective amount of 10-500 mg of the compound is administered to the subject.

44-65. (canceled)

66. A method of activating 5HT2A receptor in a subject comprising administering to a subject an effective amount of a compound having the structure: 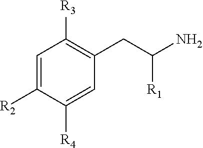
wherein
R₁ is —(C₂-C₁₂ alkyl), -(alkenyl), -(alkynyl), -(cycloalkyl);
R₂ is H, halogen, —NO₂, —CN, —CF₃, —CF₂H, —CH₂OCH₃, —CF₂CH₃, —CF₂OCH₃, -(alkyl), -(alkenyl), -(alkynyl), -(cycloalkyl), -(cycloalkylalkyl), -(heteroalkyl), -(heterocycle), -(heterocycloalkyl), -(aryl), -(heteroaryl), -(hydroxyalkyl), -(haloalkyl), -(alkylaryl), —OH, —O-(alkyl), —O-(alkenyl), —O-(alkynyl), —O-(haloalkyl), —O-(aryl), —O-(heteroaryl), —OCF₃, SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —N-(alkyl)₂, NH-(aryl), —NH-(heteroaryl), —CO₂H, —CO₂-(alkyl), —C(O)—NH₂, —C(O)—NH-(alkyl) or —C(O)—NH-(aryl), —SO₂CH₃ or —Si(CH₃) ₃;
R₃ is —OCH₃, —OCH₂CH₃, —F or —Cl; and
R₄ is —OCH₃, —OCH₂CH₃, —SCH₃, —F or —Cl,
or a pharmaceutically acceptable salt thereof, so as to thereby activate 5HT2A receptor in a subject.

67. The method of claim 66, wherein the compound has the structure: 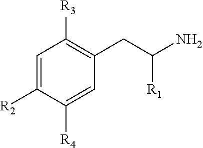
wherein
R₁ is —(C₂-C₁₂ alkyl), -(alkenyl), -(alkynyl), -(cycloalkyl);
R₂ is H, halogen, —NO₂, —CN, —CF₃, —CF₂H, -(alkyl), -(alkenyl), -(alkynyl), -(cycloalkyl), -(cycloalkylalkyl), -(heteroalkyl), -(heterocycle), -(heterocycloalkyl), -(aryl), -(heteroaryl), -(hydroxyalkyl), -(haloalkyl), -(alkylaryl), —OH, —O-(alkyl), —O-(alkenyl), —O-(alkynyl), —O-(haloalkyl), —O-(aryl), —O-(heteroaryl), —OCF₃, SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —N-(alkyl)₂, NH-(aryl), —NH-(heteroaryl), —CO₂H, —CO₂-(alkyl), —C(O)—NH₂, —C(O)—NH-(alkyl) or —C(O)—NH-(aryl) or —Si(CH₃)₃;
R₃ is —OCH₃, —F or —Cl; and
R₄ is —OCH₃, —SCH₃, —F or —Cl;
or a pharmaceutically acceptable salt thereof.

68. The method of claim 66, wherein the method is for treating the subject afflicted with Parkinson's disease, dementia, Alzheimer's disease, attention deficit hyperactivity disorder (ADHD), schizophrenia, depression, bipolar disorder, anxiety disorder, obsessive-compulsive disorder (OCD), stress disorder or a substance use disorder, or
or treating the subject afflicted with opioid withdrawal symptoms,
wherein a symptom of substance use disorder is opioid withdrawal or mitigation of relapse to opioid use or SUD, or
for treating the subject afflicted with cluster headache, diabetic retinopathy, dry eyes, macular degeneration or glaucoma.

69. The method of claim 68, wherein the substance use disorder is opioid use disorder, alcohol use disorder or stimulant use disorder including nicotine use disorder,
wherein the substance is an opioid,
wherein the opioid is morphine, hydromorphone, oxymorphone, codeine, dihydrocodeine, hydrocodone, oxycodone, nalbuphine, butorphanol, etorphine, dihydroetorphine, levorphanol, metazocine, pentazocine, meptazinol, meperidine (pethidine), buprenorphine, methadone, tramadol, tapentadol, mitragynine, 3-deutero-mitragynine, 7-hydroxymitragynine, 3-deutero-7-hydroxymitragynine, mitragynine pseudoindoxyl or tianeptine, or wherein the opioid is fentanyl, sufentanil, alfentanil, furanylfentanyl, 3-methylfentanyl, valerylfentanyl, butyrylfentanyl, β-Hydroxythiofentanyl, acrylfentanyl or carfentanil; or
wherein the stimulant is cocaine, amphetamine, methamphetamine or cathinone and its derivatives, or wherein the stimulant is nicotine,
wherein the risk of relapse to the use of opioids, alcohol or stimulants is reduced, or
wherein self-administration of an opioid, alcohol or stimulant is reduced,
wherein the effective amount of the compound administered to the subject does not induce a stimulant effect, or
wherein the effective amount of the compound administered to the subject does not induce a hallucinogenic effect, or wherein the effective amount of the compound administered to the subject does not induce a stimulant effect and a hallucinogenic effect,
wherein the subject is a mammal,
wherein the mammal is a human.

70-80. (canceled)

81. The method of claim 66, wherein the effective amount of 10-500 mg of the compound is administered to the subject.

82. The method of claim 66, wherein the compound has the structure: 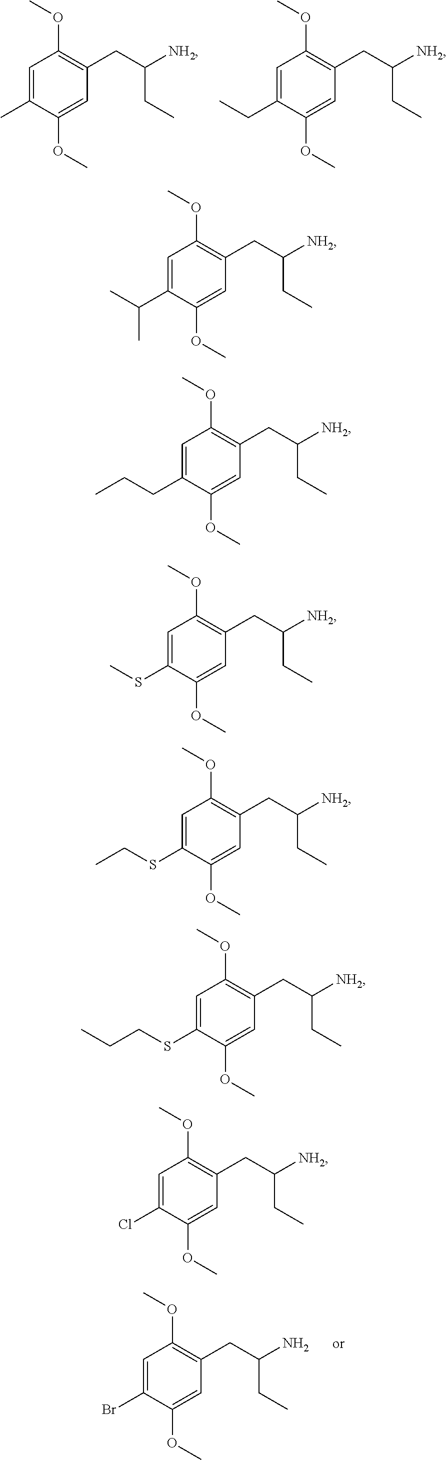 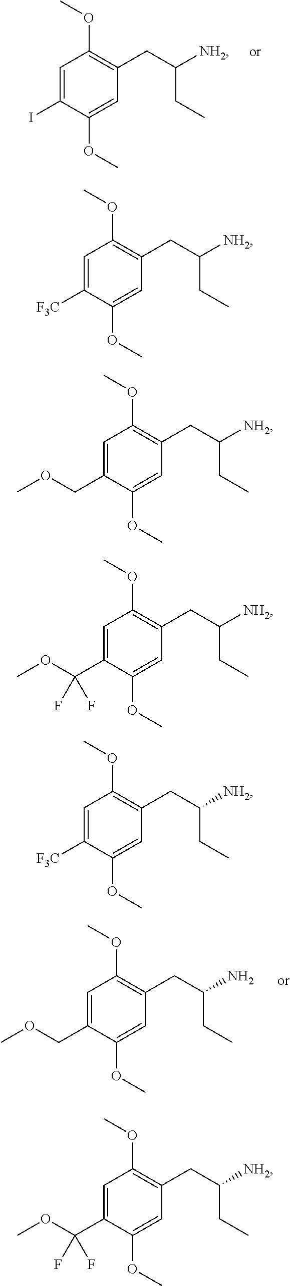
or a pharmaceutically acceptable salt thereof.

83. (canceled)
